SEQUENCE LISTING (14 sequences)

SEQ ID NO: 1 - protein, 14 aa, Homo sapiens
SVTEQGAELSNEER

SEQ ID NO: 2 - protein, 814 aa, Homo sapiens
MDAAFLLVLGLLAQSLCLSLGVPGWRRPTTLYPWRRAPALSRVRRAWVIPPISVSENHKRLPYPLVQIKSDKQQLGSVIYSIQGPGVDEEPRGVFSIDKFTGKVFLNAMLDREKTDRFRLRAFALDLGGSTLEDPTDLEIVVVDQNDNRPAFLQEAFTGRVLEGAVPGTYVTRAEATDADDPETDNAALRFSILQQGSPELFSIDELTGEIRTVQVGLDREVVAVYNLTLQVADMSGDGLTATASAIITLDDINDNAPEFTRDEFFMEAIEAVSGVDVGRLEVEDRDLPGSPNWVARFTILEGDPDGQFTIRTDPKTNEGVLSIVKALDYESCEHYELKVSVQNEAPLQAAALRAERGQAKVRVHVQDTNEPPVFQENPLRTSLAEGAPPGTLVATFSARDPDTEQLQRLSYSKDYDPEDWLQVDAATGRIQTQHVLSPASPFLKGGWYRAIVLAQDDASQPRTATGTLSIEILEVNDHAPVLAPPPPGSLCSEPHQGPGLLLGATDEDLPPHGAPFHFQLSPRLPELGRNWSLSQVNVSHARLRPRHQVPEGLHRLSLLLRDSGQPPQQREQPLNVTVCRCGKDGVCLPGAAALLAGGTGLSLGALVIVLASALLLLVLVLLVALRARFWKQSRGKGLLHGPQDDLRDNVLNYDEQGGGEEDQDAYDISQLRHPTALSLPLGPPPLRRDAPQGRLHPQPPRVLPTSPLDIADFINDGLEAADSDPSVPPYDTALIYDYEGDGSVAGTLSSILSSQGDEDQDYDYLRDWGPRFARLADMYGHPCGLEYGARWDHQAREGLSPGALLPRHRGRTA

SEQ ID NO: 3 - RNA, 21 nt, Artificial sequence
  siRNA for knockdown of CDH15
aucgccgacuucaucaaugau

SEQ ID NO: 4 - RNA, 21 nt, Artificial sequence
  siRNA for knockdown of CDH15
cacagcccucaucuaugacua

SEQ ID NO: 5 - RNA, 21 nt, Artificial sequence
  siRNA for knockdown of CDH15
cccgaucagcguauccgagaa

SEQ ID NO: 6 - RNA, 21 nt, Artificial sequence
  siRNA for knockdown of CDH15
caggacgaccuucgagacaau

SEQ ID NO: 7 - RNA, 21 nt, Artificial sequence
  siRNA for knockdown of THBS1
uacgaauguagagaucccuaa

SEQ ID NO: 8 - RNA, 21 nt, Artificial sequence
  siRNA for knockdown of THBS1
uagcugauuaacccauguaaa

SEQ ID NO: 9 - RNA, 21 nt, Artificial sequence
  siRNA for knockdown of THBS1
ugcguuggugauguaacagaa

SEQ ID NO: 10 - RNA, 21 nt, Artificial sequence
  siRNA for knockdown of THBS1
ccgaguggaccuccuguucua

SEQ ID NO: 11 - DNA, 20 bp, Artificial sequence
  Primer 1 for CDH15
ttcatcaatgatggcttgga

SEQ ID NO: 12 - DNA, 20 bp, Artificial sequence
  Primer 2 for CDH15
ctggacaggatggagctcag

SEQ ID NO: 13 - DNA, 17 bp, Artificial sequence
  Probe for CDH15
agtgtgccgccttacga

SEQ ID NO: 14 - DNA, 23738 bp, Homo sapiens
acttgcgctgtcactcagcctggacgcgcttcttcgggtcgcgggtgcactccggcccggctcccgcctcggccccgatggacgccgcgttcctcctcgtcctcgggctgttggcccaggtaaggcatcggcacctgcgggggtccccgctgcctccctcgacgctgcgggacagtgtcttcaactgcagccgcacaggtctcccccagaaccactggccctggtcgcctttgggggcctgtgagcggagtggctccgggtgggtccctgggctgggggcagcaccccaggggttgtggggagtgagtgtaaccaaacactccatctggtggaatcacccccaggactgcagagtgctgctggggtcagggaccccaggcaggggccacccaggtagccgtgccctctctttggaggaaccgctgccggggtctgggggttgccagtcttgagtggagcagagagaggggggagcggcaggagtgcccctctctggggggctgggggccaggtttctatagggactccctgcggtgggcacaggacccctggctagtcttgggttttgtccaaaccccccaccccaagttcctggccagcaggggtggctgcaggccctgcccccaggaactcaccctcacggctacttctcgattgggagggcccagcccgagccaggtcgcaacacacggccttgcagagtggcgtgtggcagccctgggggctctgttcctgggcttcacctgggaccctgccgcagctgcctggggtgatcaggcctgggggcccagcccccagaggctgctggactctgcccctaaaaatcttgtctggactgctggcaccgtggtctctccaacgtgggagcaagccagagtggaaggaagcccaggcctcagccccccacacccgacatgggtactgcctagctggagccacgtctcctcacaccctagggggacctagacatggaggatgtggcctcagtggcttcagcccccagggactgggctgggctggggcatggccggctgtggttgggacgtcactgggtacaagagggcagcctctctctggccaattcgagagagaatgcacataaagggcttagcggtgctcacagtattcattgctcttgtggtcctggggtggtcagggagtgctggacacagtgagtaagggaagccactcctttcccagcccccactgaaggggcttcctgagtcccctcattcccacaggcctcccagctccagctggctccaatttcagcccctggcagctgaccaggcgcctggccagccagaccctccagcagctgcccctgagggcaccacggctcctgccacccccgactcccccatctggagacagtggtggggggagacagtggctcagcatgcgtgctcaccccccggccctccacccctcagccctctgccggggagcgcaggctggggggctccatgcctccagggcccttccccacctgctctggggacagggtttcttccctggcaccccaacccagggcatggtggtggggaggctggcgcctgtgaaaacattcctccagacctcttcctttggatttgggagtgtctgcctttcaaaaggccccagggggctctgtggcaggtggggaggcaggcaggacctctccctcctcagaagccttccctctcactgtcccttcttttctcgcccagggctgggggacaataaccccctcccccgacagctggaggctgggggaggtggtggaggtgggggccagatggcaaggggactatcgccaaggctcaaattcactccactgtgaggcaaaatatgtcagtgtcaggaccctgcctgcccctcccccagcagctcagctttcaggactgtgagctctcctgggcagccttggggtcctgggccgcctcctggtgagcccccaggcaggcagcgtgggcccatcagggccttctacaagtgaggggttgctcccctgctcgggccacctctgattgctgagctcactcagccccacccggagggtgtcttcagaactggcccctctcagccctgctgcacctacaccatccctagttgctggttctacacctgggaccccaccccagctgggaggcggctgaggtctggggcagcacacttgactcacagagaaccccagcctgctgggaaggtccccacttctgcatctgggttttgggagccccttggtctctgggagacagtcagcagcaccctgggggccaggcaggggctcttagctcgggtgactgtgaccaaggccaagaagggaagtggctcccaggccactgttcttcccacagtggaaggacagccccagacaccgtgctttggaggggacagctgcctccatgcctcttcttccacatcccttctctgaagcccgcagcagctagagagggtggggcccagggaagatgctccagccgctctgggccaggccaacagctggggccattcctggcatgcctggccatccaggaacggggtggccccagattccagcctgggggaggggttgaagcctgagaagcttgaggggccttcgcactctcttcccaggcaaacccacccacacgccaccaagcgtgttcctagaatctcgtagcacccttccttggaaaaccgggggagcaggagtgcaaggtcggccccagaaccgctcctcgttcccgtctcgcagtggtgttcgtaaaccccattcccacctcgcagtggtgtttgtagactgcagcaagcgcccagtgtgtgcagagctttcttacacagcagccccttggggttgagtgtcaggctaggagcagacctcaagagaggcgttaagtcccaggagcttggctgcagggcggtggccccagacgtctcaagggcttcgaggtggttccctccaggctcttggctaagggtccagtcgaggatgagcttgggggtgtcattggggaccccctgtggggaagcaggtcactaatgtccatgggtcttgcctggcttgggtgggggacagagaggagtggacgagtgtccacaaaatccacctcccagggtgcttgcgtcccctaccccaggtgcagggctgtgggctggctgcgttgatgtggctttgtcctgggcaccggatgtcccgatctccccaggacggctcccaggccctccctccactggccacgtcttctgggacggggatggggtgggaggacaaagagccagctttgagaacgcccccgagagccggaagtgggccgtggccagcctgctcacccacacccacattggccgtgatctgggcaagtgagccctgccaaggctctgtggctgctgtgacctggtgaactcaggctgtggacaggcctgggcccaggactcccagcctctggcttgaccacacccggcccagcgtgactggagggtctccggtggaggcagctggaggttgggtgaacagggctggcccaggtttggtggcagctgggcagggaggggatggaaacgtctgcccgggtgggggtggcccctccacctcagccgtgtggggttcctagagcctggggtctccctgccccatgcccatgcctagcccaaggccggtgtggctctgtggctggatttccttcagagcatgcggttcagagcctaatctgggtctcagaccccacgtgactcctggctctgtctgtgtggctttgagcaaatgcctcaggctctctgaacctcactgcccagagctgggaggtgaagggaccaccacaggctcccggggggttgggaggaccaaaggggagggggagagggaggtgcaaagctgcccagccacggccggaaacagaccctgtgcccaccgaggctgatgaaggccccatgctgggcaggcaggtttccacttggcaggagctgccctgccctggaagtggcttgtgctctgtgggtgctggcaggctccgaggccagggccgccatctgtcagctggcagagagcttccccggcagggtcccctgcctgggagcctgaggaacccccaccccatcctctccctgcttcctgccaaagcgtttgctactctctggactcccaggaggccatggaggaggggactcaggtcctgctggccgaggaccaagaaccccagggaaggtggccatggtttggaggctgctgccgctgcctgtggggacctctgtgtgctgggccagccctgggcacaggacacagtcagagcgagaggccccagtgccccatctcagggtgggcagatgggcagcctgggccttgctcgtccacacagggtaagcggccggtggtggaggacagaggccaagcctaatgggggttcggcctgacaggcttcctggccatgccaacatctccttcccggcccccgcccactcagacttaccagcccccaaccgctgcctcctctcaaggccacatgcccccacgtcccggcccctgcctagactgaagtctcggcaaagccagcaggagcaggcggcactggagggtgaggcattggcacggggctgaggagagcgggcagcctcggagccagcaggggcgtggtggtcagagtagctgtcgacacctgtggttttgcagggggaggggcggcccagctggcccagctctgaccccccggttctcggtgctccatttcatggggccccaccctgcctggagcgggtgcccccttccagcggggagctgggtgaggcctcccacgcagggtcccgctcaccagggccgactcggtgctccctccgctcgcacaatgtggggagctgttctaggtgttggtgtggcgtggccggcacacctctgtagtaggcacaggacttggtgccggctggggagtcccgtgagcctccctagaccccagcccacctgctgtccagatgaaggagctcaggagcaggccgcgaagacggtgatgtggagatggggaaggagctcaggagcaggccgcgaagatggtgatattaagatgggggccctgtctgaaaggggagctgaggagtttgtgtgtggtgggtgatgtttggggcctgctctgctcccagactccccatcgcctgcctgggacccccagcacctcccacccgtccccctgcccgacatggcagcccagcagcctgagggcttgtcctgggtgggggtgccccagagagcatggcccggggtagggggtctctgtcccctcccagccgggtggtgcaggaggctgccccgtctctgcgttagggtctgttccttggggagctgctgccacccccaccacaaccctggccacagggtcctggaagcctcccaccctgcccaccccaactcagcatcttcctctgtagcccctgtcactgggacgatgcagacgccacaccctcactacacgtggtaccggtggggggcgggaagggcctcgggtctctcacccccacagccctgcagtggggggctgggaaggtcggggtaccctcgccaaggctgattggggctgtgagcgctgggcatgtggggctgggtgctggccacacatggggccttaaccacaatgaggtgaaaggggaacacagctcctcacttttgccaggcacatttcaagggctcagcagccccatgtggcctgtgggtcttggatggtgaatatctagaacattctggcagctaggagagcgctgttgggtgggagggggcctccagggaggagggtcacccgagagaaaggccgggagcctggcgacccccaggactagggaggcacagacagctcctagccaggcccggggcacaggggacggggagagggcacaggggatgggagagggcagccctgtttcctcctccctcatctccctctcccctgcctctcccccagccctggcccctgctgccctcaccaagttcaggcagactctgtgccccaacgagctgcaccaggcatcagcctgaccggggagctcgagggtgacccccatgtccgtgccgagccgagtggggtcagcccagagccatgtcctgtcagctgcggcagcttccaccaggctgtgctttagggtcaggggtctttcaggaagagccatgcagcccacccagaaacactgactccccaggctcgggggtcccaagcacaggtgtcctggggatcccaggacaggcaggacccgccgtctctaagcttccctttgaccaaggtggggccgggcaccccaggagggaggcccaaggcccaggctaggagctgggtacggtgaccgcatttttaaaaccagaaaattcaggatgtgcttttggggtcactacgagtgcccctctccacctggctcttggacttgggtcccttctctaggcctcagtttccccatctgaggggtgggtgtggggctatctgtggtcacaaaagttgctgaccgtcaggacctcgggagaatttgggggcagccacctccactttggggtctttaccggcctgagggtcgtcccggccctggagtagggtggggtgcacagcatctccctctgcaccccacactctgcccaggccccatccccgagcaccagggcttcccgtgtgcattacaaacagtgcgtcccagcccctgatgtgggagaggaggagatgggcacaccagggtcccagcagctgaagtattggggtgaagagatgaagattttaccgaggtaccctgttctgggagaagaagagtgaggctgggggtggggctgcccctctgtccccagccatgtccctcttggctgtgcagccacaacaagccccaccccacccagccccaccgaggcagccctggcccctgcccagtcagtctcaggaacccgcaccaccagcgttggctgcccagccctgacgtgttgggacctgacagaccagcagcagctgcaggccggttcagccacacagcacagtccagttgggggccgctgtatgaattacggatacatctccagctgctcttgatggactggagctgaactctcgcatcccggggttcagttcccacaaagcagcctggagaggtggagggggacgggcactttcctgcaggcacctcccctgccgctcccccggaccctccacagccagcccactgtgcccacaggcaccgccactggcttcttggggtcattccgtgtcaggaccccacgctctccccagccctggctggcccaggggctttccgtgtgcaccgctaccctggcacctggggcgaggctgtgtccccgccgggtgaatctcaggtgtccctcccagccagaggcctggaaccctagcgctacctcccagtggcccatgcactcgccaggggctgcagacagcctggctgctgagtgggcccagtaggagactcgggggcagctggatgggggctttgagggccagtacttggggtgtggccaccaaggccctggctcagtccgacgggggcggggctcacgtgcctttgcccttgtgctacgcagccccgtgtgtggaagccgccttgcgccaatgggcaccgacccgtgggctgagggaaacactgcgccccgtggcctcctcaccacccacagctcccagctgcacgctgccgcccagggctccaggagacggtactgtgggacgcatctgtctttgttgcagagcctctgcctgtctttgggggttcctggatggaggaggcccaccaccctgtacccctggcgccgggcgcctgccctgagccgcgtgcggagggcctgggtcatccccccgatcagcgtatccgagaaccacaagcgtctcccctaccccctggttcaggtgagcaggtggagggggcaggaggggagaaaggggtaggctggtccccagtgggcctccctcattctctaaaggtctcctgggagccagcggggccccatttcaggacagagctgaggcaggactcgcccaccttgccagggctctgggcttccaacctgggtctagagcagggctcaaaccctccccattgccccaggccacaggggagtccagagctgctcccaccccacatgcgcctcgggtggacatactccacgttaggctgcacgctcgggcgtgatttttgtgtgcgtgttcgccgacgctgagtagatggaggtgaaggtgaactgccttgcgtgggcctcagcttcacatacccaaaatcgccccagagaccagggccctgaggacaccaggagctgagcctgaaatgaggctgcaagacggggcacccttggggccacaggctgagctgtccccagcccagcacagctcctcattgggtaccaggatccgtcctccagtcctaggagacttagacctgccctgctgtcagctggggaggggcctgaggggctgcagagagggcagaggcccccgcccggagcagctctccccaaacccatttcctgccccacagatcaagtcggacaagcagcagctgggcagcgtcatctacagcatccagggacccggcgtggatgaggagccccggggcgtcttctctatcgacaagttcacagggaaggtcttcctcaatgccatgctggaccgcgagaagactgatcgcttcagggtgcggagctgcgtggtcggacctgtgcccctcaagcaggcctggtggaaagccagtgcccctcctccccaccaggcttctcctccccgctcggtgggcttcaggccagactgcaagatccaggcacccttaagtgagggggcaggtacaggggtttgggaccgaggccctgcagccggaggaggcaactgttgagggttatgtgcccatttaactggagggcagacagaggtcccgtggaggagcggcttggttctgccagggaggagcatgctgcagccgctgtagcttctgtggctcaaccacagccagcaacccactggtcctgggagctcaggagtgtcgtgaagaatctctaaatagtgttgtttttgttttttgtttttgagatggagtctcgctctgtcgcccaggctggagtgcagtggcgggatctcggctcactgcaagctccgcctcccgggttcacgccattctcctgcctcagcctcccgagtagctgggactacaggcccctgccaccatgcccggctaattgtttgtatttttagtagagacggggtttcactgtgttagccaggatggtctcgatctcctgagtgagccaccgcgcccgacctttttttttttttgagatggagcttcactctaccgcccaggctggagtgcagtggcacaatctcagctgactgcaatctctacctccccagttcaagcaattctcctgcttcagcctcctgagtatctgggattacaggctccaccaccgtgcccagttaatttttgtatttttagtagagatggggtttcaccatgttggcctcccaaagtgctaggattacaggcgtgagccaccacacctggccccaaaataatgttgatcccagtggccgccactgttctgggcagtggggttagtcagggaagaaaccactgatgccccttgggggctgggattctcagagaaggggcagtgagcgccgggcgcggtggctcacacaatcccagcacttcgggaggctgaggcaggaggatcgcttgaagccaatagttcgagaccaggctgggcatcaaagtgaggccttgtctcaacaaaagataaaatggtaaaacaaaaaagataaaatggtaaaataaattagccagcggtgttgctgcgtgcctgtggtcccagctgcttggaaggctgaggtgggaggatcccccgagaggtcgacactgccgtgcgccgtgattgcgccgctgcgctccagcctaggtgacagaggaagaccctctctcaaaaaaaaaagaagaaaagaggaccgggtgcggtggctcacgcctgtaatcccagcagtttgggaggccaaggcaggcagatcatgaggtcaagagatcgagaccatcctggctaacatggtgaaaccccgtctctactaaaaaatacaaaagttagctgggcattctagcgtgcgcctgtagtcccagctactcaggaggctgaggcaggagaatcgctggaacccgggaggcgaaggttgaagtgagctgagtcctcgccactgcactccagcctggtgacagagtgagactctgtctcaaaaaaaaaaacagaagaagaaagaagaagaggaggaggaagaggaggagaagaagaaagaagaagaagaaaaaaaagggcagtgggagcaagccctagagcccagaaaaccagaaccccgtctgcactcccttcaggcgcctgccctcccccagcctcccctcccccagcctgccctcccccagcctgccctcccccagcctcccctcccccagcctgccctcccccagcctcccctcccccagcctgccctcccccagcctgccctcccccagcctcccctcccccagcctgccctcccccagcctgccctcccccagcctgccctccccaagcctcccctcccccagcctgccctctcccagcctgccctctcccagcctgtcctcccccagcctgccctctccaaaatgtcattttgggctgggcacaacggctcatgcttgtaatccgagcactttgggaggccgaggcgagtggatcacctgaggtcaggagtttgagaacagtctggccaacatggcaaaaccccatctctactaaaaatacaaaaaattagccgggtgtggtggcacgcgcctgtaatcccagctagttgggaggctgaggcaggagaatcacttgaacccaggaagtggaggttgcagtgagccaagatcacaccactgcactccagcctgggcaacagagcgagatgccatctcaaaaggtaaaaaaaaagaaaagtaactttgtacttgatgtggggaaaggacgcttcctgcctttgcctctgcaggaagcaaaatttccttttatttgccccaaatcctcctttagactttgagaaccagtccttgtatcccttacatcaaaagttattagagagtatttctcacttggaaactgtaattgtgcccattctccgaaaggatgggaggcagtttaaaatgtcatgtgcacaggggaattaaacttcaggaggaggccaggcgcagtggctcacccctgtaatcccagcactttgggaggccgaggcgtatcacctgaggtcaggagtttgagaccagcctggctaacatgatgaaacctcatttctactaaaaatacaaaaaattcgctgggcgtggtgacgtgcacctgtaatcccagctacttgggaggctgaggcaggagaatcgcttgaacccaggaggcagaggttgcagtgagctgagatgtcgccattggactccagcctgggcaacaagagtgaaactctatctcaaaaaaaaaaacaaaaaacaaaaacataacttcaggaggaacaaagaccacagagagacagggagagagattaggaaccctctgagagccttactttagtcacacgtgaagctgtgagcttcctggctgccaaggcaagcagggaaagtggcacctgccctgtctgctaagggaaggtggctcccgttcagggacgtcctcagtcactgtgacagatgccccaccctgtgctgtttctcgcctgtttaagctggtgtttccagctggagacaaagtctgaacgtgctgtctctttcgcatggccctaacgggagccacagaaatttgggggccacagagccagcccttgctctatgtttgaacagctaagagcgtttgccctggacctgggaggatccaccctggaggaccccacggacctggagattgtagttgtggatcagaatgacaaccggccagccttcctgcaggaggcgttcactggccgcgtgctggagggtgcagtcccaggtgagacaggaccacagccccgggccgggaggggctgcaaggaagggctctgtgaagtccaggacttcccttaagcaagaattccagaggcccctcagagtctaaaaataagtaaacaagtctccgaggcaggtccgtttccacaggtcaactcctgccacttcccattccaacataaatttcaaacccaaaaatctgagcctgggagtggaggtttttcaggggtcttgttctcccccaaaagcaaatgacttctccctcctgctgagggggccatctggagccaggcacactccatcctcaccctgtactcacatggggtccagagcacccacaggcatctctaggttgtgattcaccctaaacccaggctgacctgtaagctccagccacggggactcgctcccacctccctgggggccggtggtaaagacctggtggaaaggcacaggccccctcgtgtctgaggctaccaaggactgtggccaggcctggcccaggctcaggcttggaggctcatatcccgcctcagtttccttcccgtgtctgtggcagtggcctccgcgcctcgttctgccccaggtgacagacagcacgtcctagctccagctgtcccggggggaagtcacagccagccaggagctgctccccagggtggcccaaagcccgtccttcccaccccacctcggtggctaagtccacctgagctgccagaggggtcaggcctaaatctcaggagagggaaatggggggaggtgaaacctggtcctgccatatgtggtccctctagcggcaccttcgtgagtaccgcttcccacagcaggtggggcgggggctctggcctacacagtcacagtgcgcgtcctctgctcggctgtcgtgtgtcctgcacccagcctgcccgcgcatcctctgttttatcctgcccttgcatcctctgtcttatcctgtccgtgcgtcctctgttttatccttcccctgcgtcctctgcccagcctgcccgtgcgtcctctgtcttatcctgcccgtgtgtcctctgttcagcctgcccatgcatcctctgtcaccctggatgccagcagctcctggaggcccccgctagctagcccaatctgacccagcgcccttagcccctgcactgggcaggcccaggagccagcacctgacccaggtcgcatccaatgctcctgtgctccgaccccataaacagcacaagccgggcaaaaggcttcgaccacataggtggcctggacacagcacggtgggcttcaggggcagcgggaagcatctcgacctgggggttgccgcatgctggccgcctcggtgacgcaaacagcagcatggactgactgccccatctggggtcagccgtgctggaaccggggagcctgtgcacacgaggtgcccccacgcccctcacaatgcccccagcccatcggccctgtggacgttggccctcacgcctccctgtgcttcccaggcacctatgtgaccagggcagaggccacagatgccgacgaccccgagacggacaacgcagcgctgcggttctccatcctgcagcagggcagccccgagctcttcagcatcgacgagctcacaggagagatccgcacagtgcaagtggggctggaccgcgaggtgaggtggcgccccggcagctccacacccgcacggccagggcagcccatctcctgcgggtccctctgcccccagcctgcccaccccagacgctcctgtccctgccgtcactgcagagcttgcagtggccctggctcctgaggagatggcatggggtgaacccactgatgcgcagacaggagccgcgctggcccgggtgggaggggtctggtgcaagtagggcgtgaggggcccggcacagggcagggctccccaagcagccccgcccctctcagcctcatggcaacggctccccgctggtaagtaactaactccttctgtcagggcccctgttgccccttgctgtcggacttcggtgctcccggaagaccccccttgagtcagctcctctaggaagccctcctggttacactgttacgtggtctttgtccatttctgttcctgccactaaccacggttccccagggcagggacccaagctcactggtgtgctaggtgcccagcacagagtaggtgctctgtaaatgcttacccagcgcacagtaggtgctctgtaaacacccagcgcacagtggtgctctgtaaacgcttatccagcacacagtaggtgctctgtaaacgcttacccagcgcagagtaggtgctctgtaaaggcttacccagcgcagagtaggtgctctgtaaatgcttacccagcgcacagtaggtgctctgtaaacacccagcgcacagtggtgctctgtaaacgcttacccagcgcacagtaggtgctctgtaaaggcttacccagcgcacagtaggtgctctgtaaacgcttacccagcacacagtaggtgctctgtaaacgcttacccagcgcacagtaggtgctctgtgaacacccagcgcacagtaggtgctctgtaaacgctcacccagtgcacagtaggtgctctgtaaacgctcacccagcgcacagtaggtgctctgtaaacgctcacccagcgcacagtaggtgctctgtaaaggctcacccagcgcacagaaggtgctctgtaaaggctcacccagcgcacagtaggtgctctgtaaacgctcacccagcgcacagtaggtgctctgtaaaggctcacccagcgcacagaaggtgctctgtaaaggcccacccagcgcacagtaggtgctctgtaaacgcttacccagcgcacagtaggtgctctgtaaacgcttacccagcgcacagtaggtgctctgtgaacacccagcgcacagtaggtgctctgtaaacgctcacccagcgcacagtaggtgctctgtaaacgctcacccagcgcacagtaggtgctctgtaaacgctcacccagcgcacagtaggtgctctgtgaacacccagcgcacagtaggtgctctgtaaaagctcacccagcgcacagaaggtgctctgtaaacgcttacccagagcacagtaggtgctctgtaaacgcttacccagcgcacagaaggtgctctgtaaacgcttacccagcgcacagaaggtgctctgtaaacgcttacccagcgcacagtaggtgctctgcaaacacccagcacacagtaggtgctctgtaaacgcttacccagcacacagtaggtgctctgtaaacgcttacccagcacacagtaggtgctctgtaaacgcttacccagcgcacagtaggtgctctgtaaacgcttacccagtgcacagtaggtgctctgtaaacgcttaccccgggcacacaaggtgctctgtaaacacttacccagcacagaataggtgctctgcaaacgcttacccagtgcagagtaggtgctctgtaaacgcttacccagcgcacagtaggtgctctgtaaacgcttacccagtgcacagtaggtgttctgtaaatgcttatccagtgccataggctagggcagcatcacagcctcatgcacttaggatcagggcaggattctcagggccacttggggtcttcaacacccactgggtgctcccgtaggaactgagctggccaggtggcctggctcccacccctgaccagtccccatgtgccccacctgggccctcatcttctgaccctgtgccccacatccccaggtggtcgcggtgtacaatctgaccctgcaggtggcggacatgtctggagacggcctcacagccactgcctcagccatcatcacccttgatgacatcaatgacaatgcccccgagttcaccagggatgaggtgctgctgctgtccctccctcgaaagtagcccctgcttagagctgccttcccttcttgggctcctgcagaaggcagcgggcttcatgatggggcaggaggatggtgtgctttggagaaggaactccgcgtgggcgggtggaagcccaagctggaggggccccggggtgctgcgtggcagtgtgtgaatggagtcagagttgggagagaggcccttggggagaagcagcccatgggaccctctgaggccctctcgcattgcagagcaagtctccctccggcctttgctgtatgggaagggagcacgaagctgagcccacagaggaggaccatgggggcacgtgggggacagtgactcacaaaacagacaaacctgaggacacccagtgggtggggagcagcttcgacaggagcaagggagggtgttcctgctgggaggcaggagggagggtgggaggggagggtgggctgagggccctgagggccccacccatgctgtccccccagccctgctggtaactggggctgggatcccccacccagttcttcatggaggccatagaggccgtcagcggagtggatgtgggacgcctggaagtggaggacagggacctgccaggctccccaaactgggtggccaggttcaccatcctggaaggcgaccccgatgggcagttcaccatccgcacggaccccaagaccaacgagggtgttctgtccattgtgaaggtgagcggcccccggctggcacacagatgccggcagacgcagatgccgacacacacagatgcccacacacagatgccggcacacacagatgcccacacacagatgccggcacacacacatgccggcacacacagatgcccacacacagatgccggcacacacacatgccggcacacacagatgcccacacacagatgccggcacacacagatgccggcacacacagatgcccacacacagatgccggcacacacagatgcccacacacagatgccggcacacacacatgccggcacacacagatgcccacacacagatgcccacgcacagatgccggcacacacagatgcccatgcacagatgccggcacacacagatgcccacacacagatgccggcacacacagatgcccacgcacagatggcggcacacacagatgccggcacacacagatgccggcacacacagatgcccacacacagatgcccacacacagatgcccacgcacaggtgcccacacacatgctggcacacagatgcccacgcacaggtgcccacacacagatgcccacgcacaggtgcccacacacagatgccagcacacacagatgcccacacacacatgcccacacacaggtgcccacacacagatgcccacactgtccacacaccgatgctggcacacatagatgcccacacacagacacccacacacagatgcccacacacaggtgcccacacacagatgttggcacacacaggtgcccacacacagatgccggcacacacagatgcccacacacagatgcccacacacagatgcccacgcacaggtgcccacacaaagatgccggcacacacagatgcccacacacagatgcccacacacagatgccggcacacacagatgcccatacacagatgcccacgcacaggtgcccacacacagatgctggcacacacagatgcccacacacatgcccacacacagatgccggcacacacagatgcccacacacagatgccggcacacacacatgccggcacacacagatgcccacacacagatgccggcacacacagatgctggcgcacacagatgcccacacacagatgcccacacacagatgcccatgctggggtttgagccccttcctggcaggttcacatcctctgaccagctgggaccttgtgttcgtccttgggtgagagagagtgggggagggcatggagccccccagcctgaggactgggtcccaggccacgctacccatggggcttgagggccagaggcaggggacaccctccaccactttgctctgtccagccttcctcctttcatatggaatccatgtacaaaatcacagctgcacacctgtgtcagtcctggttctaccagagaaagaggccataggagacacatgcacagatatgtccacacgtgtagatgtgggtgtatttagatactgacacagtaagaggtttactgcaagaaattggctcatgcggtgctggggtctggctgggcaggtctaacgtgcagaccacaggctggagccgacgcagcagtttagaggcagagaaaatgcagttttgctctcaaagccttcacctgagtggataaagccaacccacatcacccaggacgatctcctttatttaaaatcaagtgactgcaggggctggcgccatcccccaacggccctcgctgaaatacccgcggagtatgtgcttgaaccctgggctgctccccaaccaccaccacacacaaccagcattttaataatattctttgtgccttcttgtcctgcataatttgttttttcaaggttaaaaaggggcagaaggaaagtacaggtgctctggacagacccaaggagtggctcccccatggcacctgccctcgggtgcccacacttgcgttgggcggatgacgggctgtgcttccttccctcaggccctggactatgagagctgtgaacactacgaactcaaagtgtcggtgcagaatgaggccccgctgcaggcggctgcccttagggctgagcggggccaggccaaggtccgcgtgcatgtgcaggacaccaacgagccccccgtgttccaggagaacccacttcggaccagcctagcagagggggcacccccaggcactctggtggccaccttctctgcccgggaccctgacacagagcagctgcagaggctcaggtggggctcctgaggccctgggagaggtagaggaggctaacggccccattcctgcttcgggtgcccctgatccctgggctctgagggtcagagggtgtaggggccatttgctgtgtgggttcctgaaggtctggagggtctcgagtcccgagcatgcccgtgtgtgcatgttggggtgacctcaccccctagggcaggcccaactccagcctgtgcacgccagtctgtccctgccagccgtgtccatcccaggaggcccttgctcccaagggactggccgttcgcatctcatttcactccaagcctggccctgtgtatccctgtctgtgtccacggggcccgggaagggccagcaccaactgaggcttaactaggttgccacacaattgtctcagcgccctggacagctcgcctgccctccctgagcctcagtttcctcacctcacagaaggtgctggtgactgcagctcccatcatattcacatgggttgtgtgtacatgtgtatgtgcatatgtgtgtgcacatgtgtggtacatgcgtgtggtgtataggtgtgtggggggtggtatgtgcaaggtgggggggagtgtctatgtgtatatatgtgcatgtgtgtgtgctgtgcatgcccatgtatgtgtatgtggtgtgtgcatgcatgtggcatgtaaggctgtgtgtgcgtgtgtatatgttgtgtgcatgtgtgcatgcatgtggtatgtatgtgtgtgcctgtgtgtgtgcagtgcatgtcacgcacccatacctgaccacacctgtggggccctggggtaaactcagatcccactcttcccctcccctgcatcagctactccaaggactacgacccggaagactggctgcaagtggacgcagccactggccggatccagacccagcacgtgctcagcccggcgtcccccttcctcaagggcggctggtacagagccatcgtcctggcccaggatgacggtgagcggcgccgccggcttggggctccctgacctggccttgtcccggctgagcacccctgccagtgtcggagggctctgcccatgtcgcccgggggctcagagctgcgcacccgctctgagccgactggtggggcaggctggggtgttggggtcactaagccgcggcctcctcgcctgcagcctcccagccccgcaccgccaccggcaccctgtccatcgagatcctggaggtgaacgaccatgcacctgtgctggccccgccgccgccgggcagcctgtgcagcgagccacaccaaggcccaggcctcctcctgggcgccacggatgaggacctgcccccccacggggcccccttccacttccagctgagccccaggctcccagagctcggccggaactggagcctcagccaggtcaacggtgcgctcccctcaccgccgcgctccccccatccccacgctccccccacccccacattccggcctcggacgggggcaggagggtgaggggcatgcaaacccgtggtcctgcaacaggtcccctcccgccaccccccccaccactgcatcctcccgtggggcagggttactcattgtgcccagaggacggtgggggtgggggggacccaggcccaggatctcgggatccccaccctgtctcggcgcgaggagggcaggcgaagtgggggcggcctcgggaggccctcgctcaccacaggcgccctccgcagtgagccacgcgcgcctgcggccgcgacaccaggtccccgaaggcctgcaccgcctcagcctgctgctccgggactcggggcagccgccccagcagcgcgagcagcctctgaacgtgaccgtgtgccgctgcggcaaggacggcgtctgcctgccgggggccgcagcgctgctggcggggggcacaggcctcagcctgggcgcactggtcatcgtgctggccagcgccctcctgctgctgggtgagtgagcgccccgcctccacctggaccctcggaccctcggaccctcctccccaggccgtcccctgctaaccagccacgccgcttcctccccagctccgcctcctccctaaccccgccccctcattaccagccacgccgcttcctccccagctccgcctcctccctaaccccgccccgtcattaccagccacgccgcttcctccccagctccgcctcctccctaaccccgccccctcattaccagccacgccgcttcctccccagctccgcctcctccctaaccccgccccctcattaccagcctcgccgcttcctccccagctccgcctcctccctaaccccgccccctcattaccagcctcgccgcttcctccccagctcctcctcctccctaaccccgcccctcagtaccagccacgccgcttcctccccagctcctcctcctccgtaaccccgccccctcagtaccagccacgccccttcctccccagttccacctcctccgcaacaccacccactcattaccagccacgccgcttccacccaacccgccccctgctcgccaaccccgccccctcattactagccacgccccttcctccccagctccgcctcctccctaaccccgccccctcattaccagccacgccccttctccactgccgcctcctccccaacaccacccactcattaccagccacgccgcttccacccaacccgccccctgctcgcaaaccccgccccctcgttgccagccacgcctcttccccaagccggcctctccttaccagccaagctccctcctccacaacccggccccctcctccctaacctcgcggcttcctccccaaccccggctcctccatgccagacacgccctttccccaactgccgccccctcaaccccacccctgcttaccagccttgccccgccccgccccctcctcccacctccttcgcagcccggccccctgaagtcgcgccctgtgcctggccccagcctgcgtcccctcattccccagtgctggtcctgctcgtggcactccgggcgcggttctggaagcagtctcggggcaaggggctgctgcacggcccccaggacgaccttcgagacaatgtcctcaactacgatgagcaaggaggcggggaggaggaccaggtgagggggcaggtgtgggtggggaggggtccccaaggaacccaggtcgcgggccttcttacaacaagctggccaggagcctgtacctgagacctccaccagggccacccgagggatgcctggctctgttccacctcctcgcccacaggacgcctacgacatcagccagctgcgtcacccgacagcgctgagcctgcctctgggaccgccgccacttcgcagagatgccccgcagggccgcctgcacccccagccaccccgagtgctgcccaccagccccctggacatcgccgacttcatcaatgatgtaggtgctcctggggacaccccagtacacacaggcacgcacaggtgcacacacacatgcacatgtacacacctgcacatgcatgcaagaaccggcgcctgcatgcacttatgggccgtcccagagcaccgcagaggaagatggtgtgcgggcgggagtgtggaagccccgctgccaccgtccaaactgggccctcagcctccacccgcgacgcgggtctttgcacagatggagtggcgatggccacagacccatccactgccccgtgtccaccatggagggggtgggggagccccgggagcatcccctgggcttcggtggctgtcagcggaatcagggcctccatagaagaccctctggacacagcgttaatgattccctgccatccacaggatgaatgtgcacacggggaatctggggtgtggggcgcacagggaaccctcctctgtgcctgtctgcacctgtagcacctggctcagagaaagcccccagccaaggtgtacacccccggttaggagcgtgtgtccccacgagctgagaggacagacatgcagacagagcctccgaaaggcagacgggccgttctgcggatgggggtgtccaggaggctcgggttctgccttgagtggacaagggccacggggaggaagagaccagggtgagggggagcaaccccttcccaccttgctggggcagggcagagccccgggggacgggctggccgctggcctaatactgaggaagggggtagtccgtggcaccctcttcacaaccctgctgctgtgccctcaggacgctttgcctcccctcagacctcgcccccggacgtgggcagcttccccttcctgagcccgtgccttgagctgactttgccctgggctgtggccacggcggtggggcacccgctggcccctccatgtgtcttgagctctccgggcctctttatagggcttggaggctgcagatagtgaccccagtgtgccgccttacgacacagccctcatctatgactacgagggtgacggctcggtggcggggacgctgagctccatcctgtccagccagggcgatgaggaccaggactacgactacctcagagactgggggccccgcttcgcccggctggcagacatgtatgggcacccgtgcgggttggagtacggggccagatgggaccaccaggccagggagggtctttctcctggggcactgctacccagacacagaggccggacagcctgaccctggggcgcaactggacatgccactccccggcctcgtggcagtgatggcccctgcagaggcagcctgaggtcaccgggcccgacccccctgggcctggggcagcctccttcctgtaggcgagggcccaagtctgggggcagaacctgagtgtggatggggcggccaggaagaggccccttcctgccggggtgggaagagtttctctccatcggccccatgcgggtcacctccctagtcccacctttgcctcctaccagtgaacctcatctttgtatgaaagacagcaacctcctgggtaaatctgaatgaaaaacgtgctagtctctttcatgca

(19) United States
(12) Patent Application Publication
(10) Pub. No.: US 2016/0152704 A1
(43) Pub. Date: Jun. 2, 2016

(54) CADHERIN 15 AGONISTS

(71) Applicant: THE UNIVERSITY OF BIRMINGHAM, Birmingham (GB)

(72) Inventors: Myriam Chimen, Birmingham (GB); Helen Mcgettrick, Birmingham (GB); Parth Narendran, Birmingham (GB); George Edward Rainger, Worcestershire (GB)

(73) Assignee: The University of Birmingham, Birmingham (GB)

(21) Appl. No.: 14/902,193

(22) PCT Filed: Jul. 4, 2014
(86) PCT No.: PCT/GB2014/052046

§ 371 Date: Dec. 30, 2015

(30) Foreign Application Priority Data
Jul. 4, 2013 (GB) 1312010.0

(51) Int. Cl. C07K 16/28

(52) U.S. Cl. CPC C07K 16/28; C07K 2317/75

(57) ABSTRACT

CDH15 has been identified as a receptor for a peptide which has an inhibitory effect of the migration of T cells. Agonists of this receptor have applications in the treatment and/or prophylaxis of conditions associated with the migration of T-cells, including T cell auto-reactivity, T cell mediated chronic inflammatory disease and autoimmune disease. The agonist is not PEPITEM or an analogue thereof.

The present invention relates to the identification of a receptor for a peptide which has an inhibitory effect of the migration of T cells. Agonists of this receptor have applications in the treatment and/or prophylaxis of conditions associated with the migration of T-cells.

### INTRODUCTION

Lymphocytes must move repeatedly and with precision between different tissues and organs for efficient immune function. These complex trafficking pathways must be tightly controlled, because dysregulation contributes to the pathogenesis of autoimmune and chronic inflammatory diseases. The control of T-lymphocyte trafficking from the blood into inflamed tissue is of particular importance, as these cells play regulatory roles during progression and resolution of an inflammatory response.

In type 1 diabetes (T1D), pancreatic islet-reactive T cells play a central role in beta cell destruction and thus in pathogenesis. However, the mechanisms by which islet-reactive T cells are recruited from the blood, across inflamed endothelium, and into the pancreatic islet have been poorly examined. We believe that in T1D, endogenous mechanisms that prohibit the trafficking of reactive T cells into the pancreas fail, and if such regulatory pathways could be re-established it may be possible to exclude auto-reactive T cells and preserve beta cell function. The adipocyte-derived cytokine, adiponectin, has a role to play in regulating T cell migration, but the picture is more complex than that as adiponectin's circulating levels do not seem to fluctuate in T1D.

We have recently shown that adiponectin achieves its effects on T cell migration through the induction of a novel mediator, which we believe is a peptide inhibitor of T cell trans-endothelial migration which is released from B lymphocytes. This peptide inhibitor, known as PEPITEM, is described in our co-pending patent application no. GB1200555.9.

WO 2007/127935 relates to screening methods that make use of a histone deacetylase interacting with a myosin phosphatase for the identification of novel therapeutics useful for inhibiting or inducing apoptosis for the treatment of pathological conditions, such as smooth muscle cell disorder, cardiac hypertrophy or asthma. The PEPITEM peptide is identified as one of a number of proteins that bind to histone deacetylase (HDAC7). The PEPITEM peptide is also known from: US2002164668 (A1) and US20030064411 (A1), relating to diagnosis and treatment of Alzheimer's disease; and US20040053309 (A1), relating to proteins associated with kidney response to toxic effectors. However, there is no known reference to a cognate receptor for PEPITEM in the prior art.

We have now identified the receptor for the PEPITEM peptide. Surprisingly, this is cadherin-15 (CDH15). Furthermore, we have shown that agonists of this receptor other than PEPITEM are capable of inhibiting T cell migration, thus opening up a range of additional therapeutic treatments.

### SUMMARY OF THE INVENTION

According to a first aspect of the invention, there is provided a method of treatment and/or prophylaxis of a condition associated with T-cell mediated chronic inflammatory disease, including T cell auto-reactivity, T cell mediated chronic inflammatory disease and autoimmune disease, the method comprising administering an agonist of CDH15 to an individual in need thereof, wherein the agonist is not PEPITEM or an analogue thereof.

According to a second aspect of the invention, there is provided the use of a substance comprising an agonist of CDH15 in the manufacture of a medicament for the treatment and/or prophylaxis of a condition associated with T-cell mediated chronic inflammatory disease, including T cell auto-reactivity, T cell mediated chronic inflammatory disease and autoimmune disease, wherein the agonist is not PEPITEM or an analogue thereof.

According to a third aspect of the invention, there is provided an agonist of CDH15 for use in the treatment and/or prophylaxis of a condition associated with T-cell mediated chronic inflammatory disease, including T cell auto-reactivity, T cell mediated chronic inflammatory disease and autoimmune disease, by administration of the agonist of CDH15 to an individual in need thereof, wherein the agonist is not PEPITEM or an analogue thereof.

The following statements may apply to the first, second and third aspects of the invention, as appropriate, unless indicated otherwise.

In some embodiments, the condition is selected from the group consisting of: diabetes mellitus (type I); juvenile onset diabetes; rheumatoid arthritis; multiple sclerosis; Chrohn's disease; atherosclerosis; psoriasis; inflammatory and fibrotic liver disease(s) including steatohepatitis and cirrhosis; Sjogrens Syndrome; Ischaemia reperfusion injury; transplant rejection; and uveitis.

In some embodiments, the condition is selected from the group consisting of: nephropathy; diabetic kidney disease; peripheral neuropathy; diabetic retinopathy; and cardio-cerebral disease.

The agonist of CDH15 may be an antibody. The antibody may be polyclonal or monoclonal. In some embodiments, the antibody is one which binds to a protein encoded by the CDH15 gene (SEQ ID NO. 14). In some embodiments, the antibody is one which binds to an amino acid sequence corresponding to SEQ ID NO. 2, or a homologue thereof. The antibody may bind to any epitope provided on the functional CDH15 protein, or to any epitope on a functional protein encoded by the CDH15 gene. An example of a peptide sequence encoded by the CDH15 gene is provided as SEQ ID NO. 2, although there may be variations. What is important is that the binding of the antibody to the CDH15 epitope provides an agonistic effect. This effect is typically the same or similar to that provided by PEPITEM or its analogues. Preferably, this effect is the inhibition of T cell trans-endothelial migration. In some embodiments, the antibody binds to an epitope comprising the whole or part of a sequence corresponding to residues 10 to 700, residues 20 to 675, or residues 100 to 650 of SEQ ID NO 2, or the corresponding residues of a homologue thereof. In further embodiments, the antibody binds to an epitope comprising a sequence corresponding to residues 545-616 or residues 22-606 of SEQ ID NO. 2, or the corresponding residues of a homologue thereof.

In some embodiments, the antibody is one which binds the same epitope as an antibody selected from the group consisting of: Rabbit anti-human CDH15 polyclonal antibody IgG-ab75626 (Abeam); Rabbit anti-human CDH15 polyclonal antibody IgG clone H-71-sc-10734 (Santa Cruz); Rabbit anti-human CDH15 polyclonal antibody-SAB4500040 (Sigma-Aldrich); or Sheep anti-human CDH15 polyclonal antibody-AF4096 (R&D systems). In some embodiments, the antibody is one selected from the group consisting of: Rabbit anti-human CDH15 polyclonal antibody IgG-ab75626 (Abeam); Rabbit anti-human CDH15 polyclonal antibody IgG clone H-71-sc-10734 (Santa Cruz); Rabbit anti-human CDH15 polyclonal antibody-SAB4500040 (Sigma-Aldrich); or Sheep anti-human CDH15 polyclonal antibody-AF4096 (R&D systems).

The advantage of these antibodies is that they have been thoroughly tested and are already commercially available.

In general, variants of the antibodies described herein are also envisaged, provided that they retain sufficient agonistic activity to inhibit T cell trans-endothelial migration. This activity may be greater than that provided by the action of PEPITEM itself, for instance 110%, 130%, 150%, 170%, 200%, 300%, 400% or more. However, the agonistic activity provided by the variant, may also be less than that provided by PEPITEM, for instance 90% or less, 80% or less, 70% or less, 60% or less, 50% or less, 30% or less, 20% or less, or even 10% or less, as there may be advantages to having a more measured agonistic effect on the CDH15 receptor or to using an alternative therapy.

### BRIEF DESCRIPTION OF THE FIGURES

Embodiments of the invention will now be described with reference to the Figures in which:

FIG. 1 shows the effect of PEPITEM pre-treatment of T cells and/or endothelial cells on T cell transmigration;

FIG. 2 shows the effect of biotinylation on the ability of PEPITEM to inhibit transmigration of T cells;

FIG. 3a shows the effect of siRNA on expression of CDH15;

FIG. 3b shows the effect of siRNA on expression of THBS1;

FIG. 4 shows the effect of CDH15 and THBS1 knockdown on T cell transmigration;

FIG. 5 is a spectrum showing the changes in surface plasmon resonance due to binding of PEPITEM to CDH15-FC protein immobilised on a Biocore chip; and

FIG. 6 shows the effect of anti-CDH15 antibodies on T cell transmigration.

### DETAILED DESCRIPTION OF THE INVENTION

Our previous observations have shown that the PEPITEM peptide integrates with the known mechanisms of T-cell recruitment to impose a tonic inhibition on the trafficking of T-cells during inflammation. Accordingly, any reference to inhibition of T-cell migrations is preferably tonic inhibition of the same.

We have now identified cadherin-15 (CDH15) as the endothelial cell receptor of PEPITEM. The PEPITEM pathway introduces a new paradigm into the pathways regulating the inflammatory response. The processing of a 14 amino acid peptide from an intracellular protein with no known associations to the inflammatory response, and the role of cadherin-15 as a cognate endothelial cell borne receptor for PEPITEM, could not have been predicted from any of the known pathways that regulate leukocyte trafficking.

The term “PEPITEM”, as used herein, refers to a peptide of the amino acid sequence SVTEQGAELSNEER (SEQ ID NO: 1). This sequence may be comprised within a larger peptide or protein, or a chimeric or fusion protein. Alternatively, the peptide may consist solely of SEQ ID NO: 1.

Surprisingly, we have found that PEPITEM inhibits T cell trafficking through binding to cadherin-15 (CDH15). Cadherin-15 is an 814-amino acid protein that, in humans, is encoded by the CDH15 gene (SEQ ID NO. 14). This gene belongs to the cadherin superfamily of genes. Cadherin proteins consist of an extracellular domain containing 5 cadherin domains, a transmembrane region, and a conserved cytoplasmic domain. The amino acid sequence of the human CDH15 protein is shown as SEQ ID NO. 2. Thus, references herein to cadherin-15, CDH15 or “the receptor” will be understood as meaning a protein comprising or consisting of an amino acid sequence encoded by the CDH15 gene (SEQ ID NO. 14) or a protein comprising or consisting of a sequence corresponding to SEQ ID No. 2, or a homologue thereof. It will be appreciated by those skilled in the art that the CDH15 gene may give rise to multiple different proteins through alternative splicing. Splicing may occur at the transcriptional level, giving rise to different RNA sequences. Alternatively, splicing may occur post-translationally, such that different proteins arise from the same RNA sequence. Thus, references herein to “CDH15” will also be understood to include different proteins expressed by the CDH15 gene which result from alternative splicing. Such proteins may be referred to as isoforms or “splice variants”. In some embodiments, a splice variant comprises at least 30%, at least 40%, at least 50%, at least 60%, at least 70% or at least 80% of the amino acid sequence of SEQ ID NO. 2, or a homologue thereof.

As will be understood by a person skilled in the art, a “homologue” of CDH15 is a protein encoded by a gene which shares a common evolutionary ancestor with the CDH15 gene. The homologue may be a paralogue or an orthologue. A homologue of CDH15 may have at least 70%, at least 80%, at least 90%, at least 95%, at least 97%, at least 98% or at least 99% identity or similarity with the amino acid sequence of SEQ ID NO.2.

SEQ ID NO. 2: Amino acid sequence of human CDH15 from Uniprot (entry P55291):

| | |
|---|---|
| 1 | MDAAFLLVLG LLAQSLCLSL GVPGWRRPTT LYPWRRAPAL SRVRRAWVIP PISVSENHKR |
| 61 | LPYPLVQIKS DKQQLGSVIY SIQGPGVDEE PRGVFSIDKF TGKVFLNAML DREKTDRFRL |
| 121 | RAFALDLGGS TLEDPTDLEI VVVDQNDNRP AFLQEAFTGR VLEGAVPGTY VTRAEATDAD |
| 181 | DPETDNAALR FSILQQGSPE LFSIDELTGE IRTVQVGLDR EVVAVYNLTL QVADMSGDGL |
| 241 | TATASAIITL DDINDNAPEF TRDEFFMEAI EAVSGVDVGR LEVEDRDLPG SPNWVARFTI |
| 301 | LEGDPDGQFT IRTDPKTNEG VLSIVKALDY ESCEHYELKV SVQNEAPLQA AALRAERGQA |
| 361 | KVRVHVQDTN EPPVFQENPL RTSLAEGAPP GTLVATFSAR DPDTEQLQRL SYSKDYDPED |
| 421 | WLQVDAATGR IQTQHVLSPA SPFLKGGWYR AIVLAQDDAS QPRTATGTLS IEILEVNDHA |
| 481 | PVLAPPPPGS LCSEPHQGPG LLLGATDEDL PPHGAPFHFQ LSPRLPELGR NWSLSQVNVS |
| 541 | HARLRPRHQV PEGLHRLSLL LRDSGQPPQQ REQPLNVTVC RCGKDGVCLP GAAALLAGGT |
| 601 | GLSLGALVIV LASALLLLVL VLLVALRARF WKQSRGKGLL HGPQDDLRDN VLNYDEQGGG |
| 661 | EEDQDAYDIS QLRHPTALSL PLGPPPLRRD APQGRLHPQP PRVLPTSPLD IADFINDGLE |
| 721 | AADSDPSVPP YDTALIYDYE GDGSVAGTLS SILSSQGDED QDYDYLRDWG PRFARLADMY |
| 781 | GHPCGLEYGA RWDHQAREGL SPGALLPRHR GRTA |

Surprisingly, we have found that agonists of CDH15 other than PEPITEM, such as polyclonal antibodies, are capable of inhibiting T cell migration at least as effectively as PEPITEM. The present invention thus provides uses and methods of treatment and/or prophylaxis of conditions associated with T cell trafficking. These use and methods may comprise administration of an agonist of CDH15 to an individual in need thereof, wherein the agonist is not PEPITEM.

As will be understood by those skilled in the art, an agonist is a molecule that binds to a receptor and triggers a response. It will therefore be appreciated that the term “agonist”, as used herein, refers to any molecule which is capable of binding to or acting on the CDH15 receptor such that T cell trafficking is inhibited.

In some embodiments, the agonist of CDH15 is an antibody. It will be understood that the term “antibody”, as used herein, includes any form of antibodies, including domain antibodies, single chain variable region fragments or IgA/D/E/M and especially IgG (any of subtypes 1,2 3 or 4). Indeed, where reference is made herein to an antibody, it will be appreciated that this includes biopharmaceuticals. These biopharmaceuticals may include humanised antibodies, domains and fragments of antibodies, chimeric antibodies, bi-specific antibodies, antibody-drug conjugates, non-immunoglobulin protein scaffolds including, but not restricted to adnexins, darpins, camelids, shark variable domains and non-protein domains including but not restricted to aptamers.

The antibody may be human or humanised versions of any of the antibodies described herein. If that reference antibody is not human or humanised, then a humanised or human version thereof is preferred. The generation of humanised or human antibody from, for example, murine antibody is generally well known in the art.

The terms “T cell trafficking”, “T cell migration” and “T cell transmigration” are used herein to refer to the recruitment of T cells into an inflammatory site and/or inflamed tissue, preferably by migration from the blood across vascular endothelial cells. Therefore, in some embodiments the migration of the T cells is trans-endothelial. The migration of the T cells may be the recruitment of said cells to the inflamed tissues that are affected by disease. For instance, in type 1 diabetes this would include the migration of T cells into pancreas from the blood. However, it will be appreciated that in other diseases the T cells may traffic into other tissues.

It will be appreciated that the terms T cell and T lymphocyte can be interchanged herein. In some embodiments, the T cells are auto-reactive T cells. The T cells may target the pancreas, for example the islet cells of the pancreas. The T cells may be CD4+ or CD8+.

By “inhibition of T cell trafficking/migration” it will be understood that trafficking/migration is prevented or reduced. In some embodiments, the level of inhibition of migration is such that migration is reduced by at least 50% (in terms of numbers of T cells that are recruited), at least 60%, at least 70%, at least 80%, at least 90%, at least 95% or at least 99%. In further embodiments, migration is reduced to negligible levels. Ideally, of course, no T cells will migrate but this may not be realistic and in fact, all that is required is that normal function of the target tissue is preserved and/or returned (or at east as close to normal levels as possible or desirable to alleviate the condition to be treated).

It will be appreciated that the agonist acts upon the individual to which it is administered. The individual is a mammal. The mammal may be a rodent such as a rat or mouse. Alternatively, the mammal may be a primate, particularly an ape or human.

Delivery of the agonist may be by administration of the agonist per se or as a component of a composition. The agonist is preferably a protein (which includes peptides). The agonist may be delivered in protein (including fusion protein) form or as a polynucleotide encoding the protein agonist. The polypeptide may be DNA, including cDNA, or RNA, of which mRNA is preferred. The agonist may be delivered by viral vectors (such as lenti, adenoviral or adeno-associated viral vectors, and especially if in polynucleotide form) or by encapsulation in exosomes or microvesicles, which could deliver the agonist in protein/peptide or polynucleotide form.

Although gene-guns and other nanoparticle based delivery methods are preferred, in some embodiments, the agonist or composition may be administered by injection. This may be intravenously, intramuscularly or subcutaneously. It may also be administered via a mucosa, such as the oral, nasal or rectal mucosa. It may be delivered in the form of a spray or tablet or in the form of a suppository. The agonist or composition may also be ingested orally into the stomach although this may require the provision of the drug into a pro-drug to alleviate or combat the effects of the GI digestion. The methods may comprise administering to an individual in need thereof a therapeutic amount of the agonist in any of the manners described herein. As the presence of the agonist serves to inhibit the migration of the T cells, increasing the amount of agonist that the individual is exposed may serve to further inhibit said migration.

Also provided is a pharmaceutically-acceptable composition or preparation comprising the agonist. Preferably, the pharmaceutically-acceptable composition comprises the agonist and is suitable for injection or ingestion.

Provided are methods of treatment and/or prophylaxis of conditions associated with T-cell mediated chronic inflammatory disease, including T cell auto-reactivity, T cell mediated chronic inflammatory disease and autoimmune disease. In some embodiments, the condition is diabetes mellitus (type 1). In alternative embodiments, the condition is rheumatoid arthritis. In further embodiments, the condition is multiple sclerosis. However, the condition may be selected from the group consisting of: juvenile onset diabetes; rheumatoid arthritis; multiple sclerosis; Crohn's disease; atherosclerosis; psoriasis; inflammatory and fibrotic liver disease(s) including steatohepatitis and cirrhosis; and uveitis; or that the condition is selected from the group consisting of nephropathy; diabetic kidney disease; peripheral neuropathy; diabetic retinopathy; and cardio-cerebral disease.

### EXAMPLE

### Identification of the PEPITEM Receptor

PEPITEM Regulates T Cell Transmigration through Receptor Binding

We measured the transmigration of T cells (PBL) after 6 minutes incubation at 37° C. on TNF-α and IFN-α stimulated endothelial cells (EC) using phase contrast microscopy. Cultured ECs were stimulated by cytokines for 24 hours. Subsequently, peripheral blood lymphocytes (PBL) were purified from the blood of healthy donors. PEPITEM was added to the PBL and these were incubated on the endothelial cells for 6 minutes. Non-adherent PBLs were removed by washing with cell free buffer. Video-microscope images of adherent cells were made from at least 5 fields of view for each EC monolayer. Adherent lymphocytes were either, i) phase bright (PB) and adherent to the apical surface of the endothelium, or ii) phase dark (PD) and migrated through the EC monolayer. The percentage of migrated cells was calculated by dividing the number of migrated cells by the total number of adherent lymphocytes (PB+PD) and multiplying by 100. To determine whether PEPITEM targets T-cells or endothelial cells, we pre-treated T-cells, endothelial cells or both T cells and endothelial cells with PEPITEM.

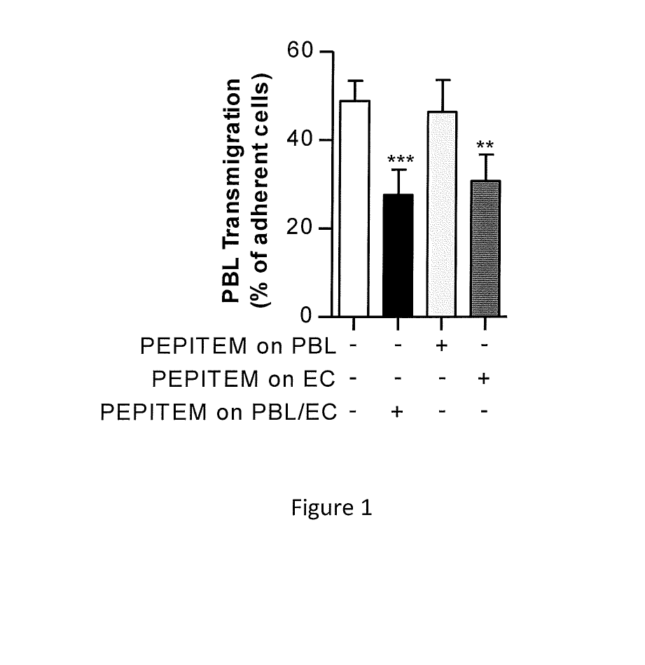

The results, shown in FIG. 1, indicate that PEPITEM is ineffective at inhibiting transmigration when T-cells alone are pre-treated, but that transmigration is successfully inhibited when endothelial cells are pre-treated with PEPITEM. This implies that PEPITEM operates by stimulating endothelial cells through a specific receptor.

PEPITEM-Receptor Binding is Uninterrupted by Biotin Conjugate

To identify the endothelial cell borne receptor, we utilised PEPITEM with a biotin conjugate on the N-terminus.

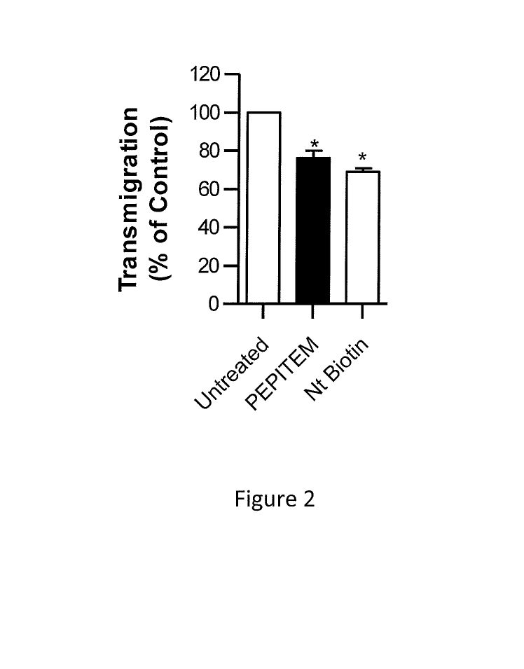

FIG. 2 shows the effect of biotinylation on the capacity of PEPITEM to inhibit transmigration. The transmigration assay was carried out as described above. Biotin was added at the N-terminus (Nt Biotin) of PEPITEM. Data are represented as mean±s.e.m and analysed by paired t-test. The PEPITEM-biotin conjugate showed full efficacy in a functional assay, demonstrating that receptor binding was uninterrupted by biotinylation.

CDH15 and THBS1 are Potential Binding Partners of PEPITEM

The biotinylated ‘bait’ was used to ‘fish’ for binding partners on the surface of endothelial cells which were co-immobilised on neutravidin columns after endothelial cell lysis. Proteins eluted from the neutravidin columns were subject to analysis by mass-spectrometry for identification.

Endothelial cells (HDMEC and HUVEC) were incubated with the N-terminus biotinylated version of PEPITEM or biotinylated scrambled control for 4 hours at 4° C. Cells were then washed twice in cold PBS and lysed with a Triton phosphate buffer (20 mM Sodium phosphate pH 7.5, 150 mM NaCl, 1% Triton X100, Protease inhibitor, all from Sigma-Aldrich). After 30 min, lysate were collected and centrifuged 20 min at 13000 rpm at 4° C. Supernatants were collected and dried under vacuum and the samples resuspended in 20 μl 8 M Urea/2% SDS and loading buffer (Sigma-aldrich and Life Technologies Invitrogen Compounds). Samples were loaded onto a 4-12% SDS-PAGE gel (Life Technologies Invitrogen Compounds) and stained overnight with Colloidal Coomassie staining buffer (0.08% Coomassie Brilliant Blue G250, 1.6% Orthophosphoric Acid, 8% Ammonium Sulphate, 20% Methanol, all from Sigma-Aldrich). Gel was detained in 1% Acetic acid in distilled water (several changes) until the background was clear. Protein bands were cut-off the gel and wash twice in 50% Acetonitrile (ACN)/50 nM Ammonium Bicarbonate (AB, Sigma-Aldrich) for 45 min at 37° C. with agitation. The gel were then incubated at 56° C. for 1 hour in 50 mM DTT in 10% ACN/50 mM AB then in 100 mM in 10% ACN/50 mM AB for 30 minutes at room temperature in the dark. Bands were washed twice in 10% ACN/40 mM AB for 15 minutes and dried under vacuum until completely dry. Trypsin was then added on the bands at 200 μg/ml in 10% ACN/40 mM AB and left overnight at 37° C. Supernatant was then collected and bands were washed twice in 3% Formic acid for 1 hour at room temperature under agitation. Supernatants were collected and pooled together after all washes and samples were dried under vacuum, resuspended in 20 μl 0.1% formic acid in 2% acetonitrile. 10 μl of the purified samples was subjected to an LC-MS/MS analysis using a gradient of 2-36% ACN in 0.1% formic acid over 30 min at 350 nL/min using a Dionex Ultimate 3000 HPLC system.

MS analysis yielded a list of proteins using PEPITEM and a scrambled control peptide (the PEPITEM peptide with the amino acids in a different order). We compared both list of proteins and identified candidate receptors that were found in the PEPITEM eluate of the column and not in the scrambled control eluate. This method identified two candidates with strong statistical scores, cadherin-15 (CDH15; Cadherin-4; M-cadherin) and thrombospondin-1 (THBS1).

siRNA Knockdown of CDH15 Restores Transmigration in Presence of PEPITEM

The effect of siRNA knockdown of CDH15 and THBS1 on T-cell trafficking was then investigated.

HUVEC were plated in a 12 well plate (87500 cells per well) for 24 hours or until about 80% confluence. The relevant siRNA were added at a final concentration of 50 nM to 83.75 μl or 82.5 μl if duplex in Optimem media (Life Technologies Invitrogen Compounds). 1.5 μl of RNAi Lipofectamine (Life Technologies Invitrogen Compounds) was mixed with 13.5 μl of Optimem and these were incubated for 10 minutes at room temperature. 15 μl of Lipofectamine mix was added to each siRNA singleplex or duplex, gently mixed and incubated for a further 10 minutes. HUVEC were washed twice with PBS and 400 μl of Optimem was added to the Lipofectamine siRNA duplexes. After gentle agitation, the mix was added on the HUVEC and incubated at 37° C. for four hours. The mix was then replaced with the classic low serum media without antibiotics. After 48 hours, HUVEC were stimulated with TNF-α/IFN-γ for an additional 24 hours before measuring PBL adhesion and migration as described previously. siRNA sequences used for knockdown of CDH15 were:

#1:
(SEQ ID NO. 3)
AUCGCCGACUUCAUCAAUGAU;
#2:
(SEQ ID NO. 4)
CACAGCCCUCAUCUAUGACUA;
#3:
(SEQ ID NO. 5)
CCCGAUCAGCGUAUCCGAGAA;
#4:
(SEQ ID NO. 6)
CAGGACGACCUUCGAGACAAU.
siRNA sequences used for knockdown of THBS1 were:
#1:
(SEQ ID NO. 7)
UACGAAUGUAGAGAUCCCUAA;
#2:
(SEQ ID NO. 8)
UAGCUGAUUAACCCAUGUAAA;
#3:
(SEQ ID NO. 9)
UGCGUUGGUGAUGUAACAGAA;
#4:
(SEQ ID NO. 10)
CCGAGUGGACCUCCUGUUCUA.

qPCR: Total mRNA was extracted using the RNAeasy Minikit (Qiagen, Crawley, UK) according to the manufacturer's protocol. Briefly, PBMC were first lysed then added to a column, after three washes, mRNA was eluted from the column with water. RNA concentration was measured using Nanodrop spectrofluorimeter (LabTech) and RNA was stored at −80° C. To convert RNA to cDNA, random primers (Promega, Maddison, USA) were annealed to 1 μg of mRNA for five minutes at 70° C., after which the following mastermix was added to give a final volume of 30 μl: 10U Superscript II Reverse Transcriptase (RT), 10U RNAout RNase inhibitor, 1× Superscript Buffer (all from Invitrogen) and 10 mM dNTPs (Promega). The reaction was run at 37° C. for one hour, followed by five minutes at 95° C. TBHS1 FAM-labeled and 18S VIC-labelled primers were bought as Assay on Demand kits from Applied Biosystems (Warrington, U.K.) to analyze mRNA. Primers against CDH15 were designed in house and bought from Eurogentec

(Primer 1: TTCATCAATGATGGCTTGGA (SEQ ID NO. 11);
Primer 2: CTGGACAGGATGGAGCTCAG (SEQ ID NO. 12);
Probe: AGTGTGCCGCCTTACGA (SEQ ID NO. 13)).

Samples were amplified in duplicates using the 7500HT Real-Time PCR machine (Applied Biosystems) and analyzed using the software package SDS 2.2 (Applied Biosystems). Data were expressed as relative expression units relative to 18S.

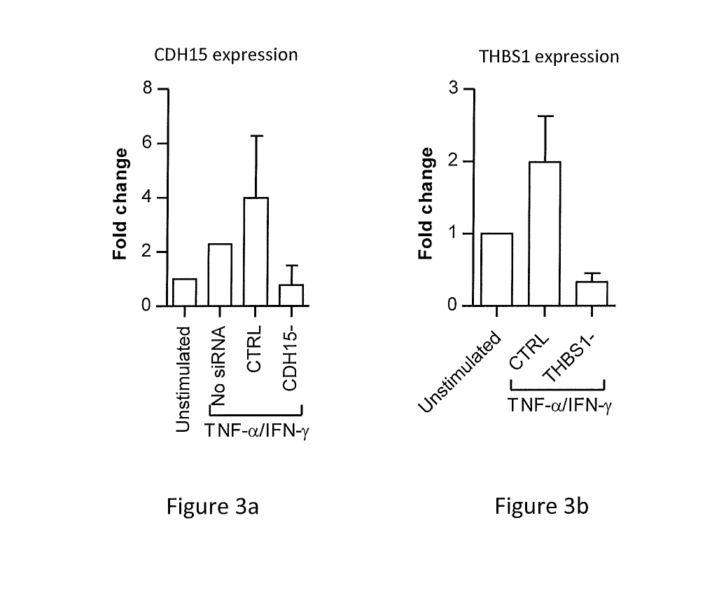

As shown in FIG. 3, CDH15 and THBS1 were efficiently knocked down in endothelial cells by specific siRNA oligomers, but not by control sequences. This demonstrated that the siRNA oligomers were effective at knocking down expression of the receptor candidates. Interestingly, cadherin-15 has not previously been described as part of the endothelial cell transcriptome, and here we show that it is endogenously expressed, as well as being up-regulated when endothelial cells were stimulated with inflammatory cytokines.

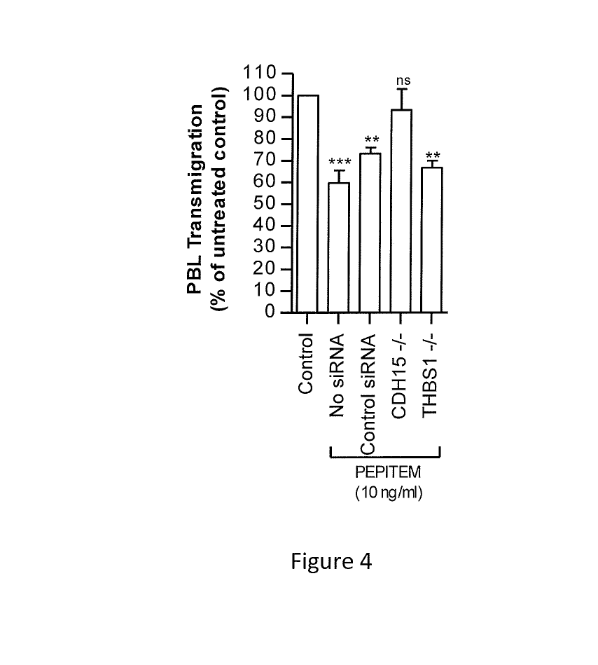

We then carried out the transmigration assay as previously described using endothelial cells where CDH15 or THBS1 were knock-down. FIG. 4 shows the effect of knockdown of CDH15 and THBS1 on transmigration. Importantly, the ablation of cadherin-15 released T-cells from the inhibitory effects of PEPITEM, whereas knockdown of thrombospondin-1 had no significant effect on T-cell trafficking. This indicates that cadherin-15 is the PEPITEM receptor, and that interaction of cadherin-15 and PEPITEM is essential for inhibition of T cell migration by the peptide. The lack of effect of thrombospondin-1 knock down serves as a good control, showing that the effects of siRNA treatment were specific to the CDH15 gene.

CDH15 Protein is Present in Endothelial Cells

Immunoprecipitation and Western Blot: Whole cell lysates from muscle cells, HUVEC and HDMEC were extracted by suspending the cells in cell lysis buffer with 50 mM Tris-HCl pH 8, 150 mM NaCl, 10% glycerol, 1% (w/v) Nonidet P-40, 0.5% (w/v) sodium deoxycholate and protease inhibitor cocktail (Invitrogen). After incubation for 15 minutes at 4° C., this preparation was centrifuged at 2000 r.p.m. for 10 minutes. The supernatant was subjected to immunoprecipitation by incubating for 30 minutes at 4° C. with protein G—Dyna beads (Invitrogen) and polyclonal anti-CDH-15 antibody (R&D systems). The beads were collected and washed three times with ice-cold extraction buffer and once with 50 mM Tris-HCl pH 7.5. Proteins that were retained on the beads were eluted using glycine and separated by SDS-PAGE 10% (w/v) and analysed by western blot with a sheep anti-human CDH15 antibody (R/D systems). Blots were then probed with appropriate horseradish peroxidase-conjugated anti rabbit secondary antibody (Cell Signalling Technology, UK). Immunodetection was carried out using the ECL plus Kit (Amersham, GE Healthcare Life Sciences, UK) followed by exposure to X-ray film for 15 min. Controls were run in parallel with application of the recombinant CDH15 (R&D systems).

Confocal Microscopy: Muscle cells, HDMEC and HUVEC were grown to confluence onto glass chamber slides (Becton Dickinson Falcon) at 37° C. They were then fixed with 2% PFA and 4% sucrose for 15 min, washed in PBS and stained with 10 μg/ml of either a sheep anti-human CDH15 antibody (R&D systems) or sheep IgG (Southern Biotech, UK) overnight at 4° C. Goat anti-sheep IgG1 conjugated to Alexa 488 (Abeam, UK) was then applied and the slides were visualized using a Zeiss confocal LSM 510 microscope (Zeiss, Gottingen, Germany) and processed using Zeiss LSM Image Examiner software (Zeiss).

Cadherin-15 expression was detected at a protein level using western blotting analysis which showed a specific band at between ˜70-90 kDa in both HUVEC, HDMEC and muscle cells used as a control. CDH15 expression was up-regulated by stimulation with TIMF-α/INFγ. Specific siRNA, but not control siRNA, dramatically reduced CDH15 expression. We also found intracellular and membrane CDH15 protein expression using confocal microscopy in HUVEC, HDMEC and human skeletal muscle cells. Low levels of CDH15 were present in unstimulated EC and CDH14 was up-regulated upon stimulation with TNF-α/INFγ. This is surprising because CDH15 has not previously been described in endothelial cells and thus represents a novel endothelial mediator of the inflammatory response.

Specific Binding of PEPITEM to CDH15

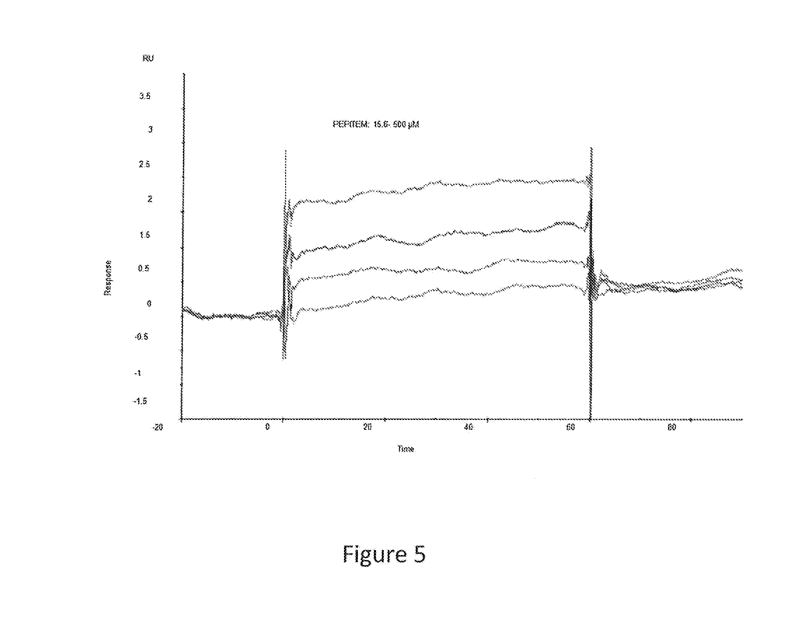

Gold-coated chips were used to analyse the interaction of CDH15 with PEPITEM in the Biocore 3000 instrument. CDH15-FC protein was immobilised on a Biocore chip. PEPITEM was perfused through the Biocore cell at increasing concentration. As shown in FIG. 5, PEPITEM was able to bind to the CDH15 protein as demonstrated by changes in SPR showing that there was a concentration dependent change in SPR. This provides further evidence that cadherin-15 is the PEPITEM receptor.

Blockade of CDH15 Causes Inhibition of PBL Transmigration

Finally, CDH15 was targeted in the transmigration experiments using polyclonal anti human CDH15 antibodies (Abeam: Rabbit anti-human CDH15 polyclonal antibody IgG-ab75626, targets N-terminal; Santa Cruz: Rabbit anti-human CDH15 polyclonal antibody IgG clone H-71-sc-10734, targets amino acids 545-615 of CDH15; Sigma-Aldrich: Rabbit anti-human CDH15 polyclonal antibody-SAB4500040, targets N-terminal; R&D systems: Sheep anti-human CDH15 polyclonal antibody-AF4096, targets amino acids 22-606) for 15 minutes at 37° C. prior to lymphocyte (PBL) transmigration. As controls we used an irrelevant sheep and rabbit isotype as well as sodium azide, which is present in low amounts in the buffer of all commercially available antibodies. The concentration of antibodies used was 10 μg/ml.

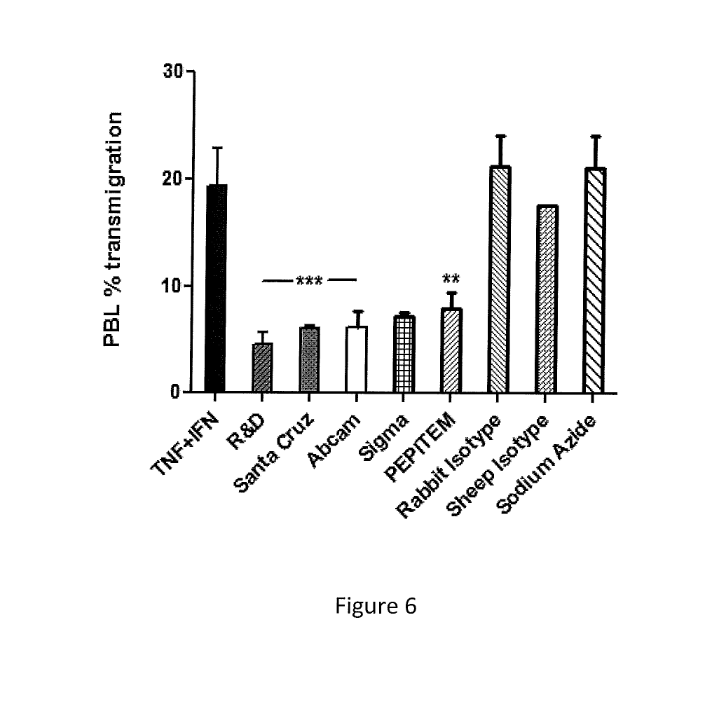

The results are shown in FIG. 6. Each of the four polyclonal antibodies tested showed greater efficacy than PEPITEM itself at inhibiting T cell transmigration. This indicates that these antibodies are agonists of CDH15. The agonistic nature of all of the polyclonal antibodies tested is very surprising, particularly since the antibodies are likely to have a number of epitope binding sites on CDH15.

SEQ ID No. 14: DNA sequence of human CDH15 gene (chromosome 16):
ACTTGCGCTGTCACTCAGCCTGGACGCGCTTCTTCGGGTCGCGGGTGCACTCCGGCCCGGCTCCCGCCTC
GGCCCCGATGGACGCCGCGTTCCTCCTCGTCCTCGGGCTGTTGGCCCAGGTAAGGCATCGGCACCTGCGG
GGGTCCCCGCTGCCTCCCTCGACGCTGCGGGACAGTGTCTTCAACTGCAGCCGCACAGGTCTCCCCCAGA
ACCACTGGCCCTGGTCGCCTTTGGGGGCCTGTGAGCGGAGTGGCTCCGGGTGGGTCCCTGGGCTGGGGGC
AGCACCCCAGGGGTTGTGGGGAGTGAGTGTAACCAAACACTCCATCTGGTGGAATCACCCCCAGGACTGC
AGAGTGCTGCTGGGGTCAGGGACCCCAGGCAGGGGCCACCCAGGTAGCCGTGCCCTCTCTTTGGAGGAAC
CGCTGCCGGGGTCTGGGGGTTGCCAGTCTTGAGTGGAGCAGAGAGAGGGGGGAGCGGCAGGAGTGCCCCT
CTCTGGGGGGCTGGGGGCCAGGTTTCTATAGGGACTCCCTGCGGTGGGCACAGGACCCCTGGCTAGTCTT
GGGTTTTGTCCAAACCCCCCACCCCAAGTTCCTGGCCAGCAGGGGTGGCTGCAGGCCCTGCCCCCAGGAA
CTCACCCTCACGGCTACTTCTCGATTGGGAGGGCCCAGCCCGAGCCAGGTCGCAACACACGGCCTTGCAG
AGTGGCGTGTGGCAGCCCTGGGGGCTCTGTTCCTGGGCTTCACCTGGGACCCTGCCGCAGCTGCCTGGGG
TGATCAGGCCTGGGGGCCCAGCCCCCAGAGGCTGCTGGACTCTGCCCCTAAAAATCTTGTCTGGACTGCT
GGCACCGTGGTCTCTCCAACGTGGGAGCAAGCCAGAGTGGAAGGAAGCCCAGGCCTCAGCCCCCCACACC
CGACATGGGTACTGCCTAGCTGGAGCCACGTCTCCTCACACCCTAGGGGGACCTAGACATGGAGGATGTG
GCCTCAGTGGCTTCAGCCCCCAGGGACTGGGCTGGGCTGGGGCATGGCCGGCTGTGGTTGGGACGTCACT
GGGTACAAGAGGGCAGCCTCTCTCTGGCCAATTCGAGAGAGAATGCACATAAAGGGCTTAGCGGTGCTCA
CAGTATTCATTGCTCTTGTGGTCCTGGGGTGGTCAGGGAGTGCTGGACACAGTGAGTAAGGGAAGCCACT
CCTTTCCCAGCCCCCACTGAAGGGGCTTCCTGAGTCCCCTCATTCCCACAGGCCTCCCAGCTCCAGCTGG
CTCCAATTTCAGCCCCTGGCAGCTGACCAGGCGCCTGGCCAGCCAGACCCTCCAGCAGCTGCCCCTGAGG
GCACCACGGCTCCTGCCACCCCCGACTCCCCCATCTGGAGACAGTGGTGGGGGGAGACAGTGGCTCAGCA
TGCGTGCTCACCCCCCGGCCCTCCACCCCTCAGCCCTCTGCCGGGGAGCGCAGGCTGGGGGGCTCCATGC
CTCCAGGGCCCTTCCCCACCTGCTCTGGGGACAGGGTTTCTTCCCTGGCACCCCAACCCAGGGCATGGTG
GTGGGGAGGCTGGCGCCTGTGAAAACATTCCTCCAGACCTCTTCCTTTGGATTTGGGAGTGTCTGCCTTT
CAAAAGGCCCCAGGGGGCTCTGTGGCAGGTGGGGAGGCAGGCAGGACCTCTCCCTCCTCAGAAGCCTTCC
CTCTCACTGTCCCTTCTTTTCTCGCCCAGGGCTGGGGGACAATAACCCCCTCCCCCGACAGCTGGAGGCT
GGGGGAGGTGGTGGAGGTGGGGGCCAGATGGCAAGGGGACTATCGCCAAGGCTCAAATTCACTCCACTGT
GAGGCAAAATATGTCAGTGTCAGGACCCTGCCTGCCCCTCCCCCAGCAGCTCAGCTTTCAGGACTGTGAG
CTCTCCTGGGCAGCCTTGGGGTCCTGGGCCGCCTCCTGGTGAGCCCCCAGGCAGGCAGCGTGGGCCCATC
AGGGCCTTCTACAAGTGAGGGGTTGCTCCCCTGCTCGGGCCACCTCTGATTGCTGAGCTCACTCAGCCCC
ACCCGGAGGGTGTCTTCAGAACTGGCCCCTCTCAGCCCTGCTGCACCTACACCATCCCTAGTTGCTGGTT
CTACACCTGGGACCCCACCCCAGCTGGGAGGCGGCTGAGGTCTGGGGCAGCACACTTGACTCACAGAGAA
CCCCAGCCTGCTGGGAAGGTCCCCACTTCTGCATCTGGGTTTTGGGAGCCCCTTGGTCTCTGGGAGACAG
TCAGCAGCACCCTGGGGGCCAGGCAGGGGCTCTTAGCTCGGGTGACTGTGACCAAGGCCAAGAAGGGAAG
TGGCTCCCAGGCCACTGTTCTTCCCACAGTGGAAGGACAGCCCCAGACACCGTGCTTTGGAGGGGACAGC
TGCCTCCATGCCTCTTCTTCCACATCCCTTCTCTGAAGCCCGCAGCAGCTAGAGAGGGTGGGGCCCAGGG
AAGATGCTCCAGCCGCTCTGGGCCAGGCCAACAGCTGGGGCCATTCCTGGCATGCCTGGCCATCCAGGAA
CGGGGTGGCCCCAGATTCCAGCCTGGGGGAGGGGTTGAAGCCTGAGAAGCTTGAGGGGCCTTCGCACTCT
CTTCCCAGGCAAACCCACCCACACGCCACCAAGCGTGTTCCTAGAATCTCGTAGCACCCTTCCTTGGAAA
ACCGGGGGAGCAGGAGTGCAAGGTCGGCCCCAGAACCGCTCCTCGTTCCCGTCTCGCAGTGGTGTTCGTA
AACCCCATTCCCACCTCGCAGTGGTGTTTGTAGACTGCAGCAAGCGCCCAGTGTGTGCAGAGCTTTCTTA
CACAGCAGCCCCTTGGGGTTGAGTGTCAGGCTAGGAGCAGACCTCAAGAGAGGCGTTAAGTCCCAGGAGC
TTGGCTGCAGGGCGGTGGCCCCAGACGTCTCAAGGGCTTCGAGGTGGTTCCCTCCAGGCTCTTGGCTAAG
GGTCCAGTCGAGGATGAGCTTGGGGGTGTCATTGGGGACCCCCTGTGGGGAAGCAGGTCACTAATGTCCA
TGGGTCTTGCCTGGCTTGGGTGGGGGACAGAGAGGAGTGGACGAGTGTCCACAAAATCCACCTCCCAGGG
TGCTTGCGTCCCCTACCCCAGGTGCAGGGCTGTGGGCTGGCTGCGTTGATGTGGCTTTGTCCTGGGCACC
GGATGTCCCGATCTCCCCAGGACGGCTCCCAGGCCCTCCCTCCACTGGCCACGTCTTCTGGGACGGGGAT
GGGGTGGGAGGACAAAGAGCCAGCTTTGAGAACGCCCCCGAGAGCCGGAAGTGGGCCGTGGCCAGCCTGC
TCACCCACACCCACATTGGCCGTGATCTGGGCAAGTGAGCCCTGCCAAGGCTCTGTGGCTGCTGTGACCT
GGTGAACTCAGGCTGTGGACAGGCCTGGGCCCAGGACTCCCAGCCTCTGGCTTGACCACACCCGGCCCAG
CGTGACTGGAGGGTCTCCGGTGGAGGCAGCTGGAGGTTGGGTGAACAGGGCTGGCCCAGGTTTGGTGGCA
GCTGGGCAGGGAGGGGATGGAAACGTCTGCCCGGGTGGGGGTGGCCCCTCCACCTCAGCCGTGTGGGGTT
CCTAGAGCCTGGGGTCTCCCTGCCCCATGCCCATGCCTAGCCCAAGGCCGGTGTGGCTCTGTGGCTGGAT
TTCCTTCAGAGCATGCGGTTCAGAGCCTAATCTGGGTCTCAGACCCCACGTGACTCCTGGCTCTGTCTGT
GTGGCTTTGAGCAAATGCCTCAGGCTCTCTGAACCTCACTGCCCAGAGCTGGGAGGTGAAGGGACCACCA
CAGGCTCCCGGGGGGTTGGGAGGACCAAAGGGGAGGGGGAGAGGGAGGTGCAAAGCTGCCCAGCCACGGC
CGGAAACAGACCCTGTGCCCACCGAGGCTGATGAAGGCCCCATGCTGGGCAGGCAGGTTTCCACTTGGCA
GGAGCTGCCCTGCCCTGGAAGTGGCTTGTGCTCTGTGGGTGCTGGCAGGCTCCGAGGCCAGGGCCGCCAT
CTGTCAGCTGGCAGAGAGCTTCCCCGGCAGGGTCCCCTGCCTGGGAGCCTGAGGAACCCCCACCCCATCC
TCTCCCTGCTTCCTGCCAAAGCGTTTGCTACTCTCTGGACTCCCAGGAGGCCATGGAGGAGGGGACTCAG
GTCCTGCTGGCCGAGGACCAAGAACCCCAGGGAAGGTGGCCATGGTTTGGAGGCTGCTGCCGCTGCCTGT
GGGGACCTCTGTGTGCTGGGCCAGCCCTGGGCACAGGACACAGTCAGAGCGAGAGGCCCCAGTGCCCCAT
CTCAGGGTGGGCAGATGGGCAGCCTGGGCCTTGCTCGTCCACACAGGGTAAGCGGCCGGTGGTGGAGGAC
AGAGGCCAAGCCTAATGGGGGTTCGGCCTGACAGGCTTCCTGGCCATGCCAACATCTCCTTCCCGGCCCC
CGCCCACTCAGACTTACCAGCCCCCAACCGCTGCCTCCTCTCAAGGCCACATGCCCCCACGTCCCGGCCC
CTGCCTAGACTGAAGTCTCGGCAAAGCCAGCAGGAGCAGGCGGCACTGGAGGGTGAGGCATTGGCACGGG
GCTGAGGAGAGCGGGCAGCCTCGGAGCCAGCAGGGGCGTGGTGGTCAGAGTAGCTGTCGACACCTGTGGT
TTTGCAGGGGGAGGGGCGGCCCAGCTGGCCCAGCTCTGACCCCCCGGTTCTCGGTGCTCCATTTCATGGG
GCCCCACCCTGCCTGGAGCGGGTGCCCCCTTCCAGCGGGGAGCTGGGTGAGGCCTCCCACGCAGGGTCCC
GCTCACCAGGGCCGACTCGGTGCTCCCTCCGCTCGCACAATGTGGGGAGCTGTTCTAGGTGTTGGTGTGG
CGTGGCCGGCACACCTCTGTAGTAGGCACAGGACTTGGTGCCGGCTGGGGAGTCCCGTGAGCCTCCCTAG
ACCCCAGCCCACCTGCTGTCCAGATGAAGGAGCTCAGGAGCAGGCCGCGAAGACGGTGATGTGGAGATGG
GGAAGGAGCTCAGGAGCAGGCCGCGAAGATGGTGATATTAAGATGGGGGCCCTGTCTGAAAGGGGAGCTG
AGGAGTTTGTGTGTGGTGGGTGATGTTTGGGGCCTGCTCTGCTCCCAGACTCCCCATCGCCTGCCTGGGA
CCCCCAGCACCTCCCACCCGTCCCCCTGCCCGACATGGCAGCCCAGCAGCCTGAGGGCTTGTCCTGGGTG
GGGGTGCCCCAGAGAGCATGGCCCGGGGTAGGGGGTCTCTGTCCCCTCCCAGCCGGGTGGTGCAGGAGGC
TGCCCCGTCTCTGCGTTAGGGTCTGTTCCTTGGGGAGCTGCTGCCACCCCCACCACAACCCTGGCCACAG
GGTCCTGGAAGCCTCCCACCCTGCCCACCCCAACTCAGCATCTTCCTCTGTAGCCCCTGTCACTGGGACG
ATGCAGACGCCACACCCTCACTACACGTGGTACCGGTGGGGGGCGGGAAGGGCCTCGGGTCTCTCACCCC
CACAGCCCTGCAGTGGGGGGCTGGGAAGGTCGGGGTACCCTCGCCAAGGCTGATTGGGGCTGTGAGCGCT
GGGCATGTGGGGCTGGGTGCTGGCCACACATGGGGCCTTAACCACAATGAGGTGAAAGGGGAACACAGCT
CCTCACTTTTGCCAGGCACATTTCAAGGGCTCAGCAGCCCCATGTGGCCTGTGGGTCTTGGATGGTGAAT
ATCTAGAACATTCTGGCAGCTAGGAGAGCGCTGTTGGGTGGGAGGGGGCCTCCAGGGAGGAGGGTCACCC
GAGAGAAAGGCCGGGAGCCTGGCGACCCCCAGGACTAGGGAGGCACAGACAGCTCCTAGCCAGGCCCGGG
GCACAGGGGACGGGGAGAGGGCACAGGGGATGGGAGAGGGCAGCCCTGTTTCCTCCTCCCTCATCTCCCT
CTCCCCTGCCTCTCCCCCAGCCCTGGCCCCTGCTGCCCTCACCAAGTTCAGGCAGACTCTGTGCCCCAAC
GAGCTGCACCAGGCATCAGCCTGACCGGGGAGCTCGAGGGTGACCCCCATGTCCGTGCCGAGCCGAGTGG
GGTCAGCCCAGAGCCATGTCCTGTCAGCTGCGGCAGCTTCCACCAGGCTGTGCTTTAGGGTCAGGGGTCT
TTCAGGAAGAGCCATGCAGCCCACCCAGAAACACTGACTCCCCAGGCTCGGGGGTCCCAAGCACAGGTGT
CCTGGGGATCCCAGGACAGGCAGGACCCGCCGTCTCTAAGCTTCCCTTTGACCAAGGTGGGGCCGGGCAC
CCCAGGAGGGAGGCCCAAGGCCCAGGCTAGGAGCTGGGTACGGTGACCGCATTTTTAAAACCAGAAAATT
CAGGATGTGCTTTTGGGGTCACTACGAGTGCCCCTCTCCACCTGGCTCTTGGACTTGGGTCCCTTCTCTA
GGCCTCAGTTTCCCCATCTGAGGGGTGGGTGTGGGGCTATCTGTGGTCACAAAAGTTGCTGACCGTCAGG
ACCTCGGGAGAATTTGGGGGCAGCCACCTCCACTTTGGGGTCTTTACCGGCCTGAGGGTCGTCCCGGCCC
TGGAGTAGGGTGGGGTGCACAGCATCTCCCTCTGCACCCCACACTCTGCCCAGGCCCCATCCCCGAGCAC
CAGGGCTTCCCGTGTGCATTACAAACAGTGCGTCCCAGCCCCTGATGTGGGAGAGGAGGAGATGGGCACA
CCAGGGTCCCAGCAGCTGAAGTATTGGGGTGAAGAGATGAAGATTTTACCGAGGTACCCTGTTCTGGGAG
AAGAAGAGTGAGGCTGGGGGTGGGGCTGCCCCTCTGTCCCCAGCCATGTCCCTCTTGGCTGTGCAGCCAC
AACAAGCCCCACCCCACCCAGCCCCACCGAGGCAGCCCTGGCCCCTGCCCAGTCAGTCTCAGGAACCCGC
ACCACCAGCGTTGGCTGCCCAGCCCTGACGTGTTGGGACCTGACAGACCAGCAGCAGCTGCAGGCCGGTT
CAGCCACACAGCACAGTCCAGTTGGGGGCCGCTGTATGAATTACGGATACATCTCCAGCTGCTCTTGATG
GACTGGAGCTGAACTCTCGCATCCCGGGGTTCAGTTCCCACAAAGCAGCCTGGAGAGGTGGAGGGGGACG
GGCACTTTCCTGCAGGCACCTCCCCTGCCGCTCCCCCGGACCCTCCACAGCCAGCCCACTGTGCCCACAG
GCACCGCCACTGGCTTCTTGGGGTCATTCCGTGTCAGGACCCCACGCTCTCCCCAGCCCTGGCTGGCCCA
GGGGCTTTCCGTGTGCACCGCTACCCTGGCACCTGGGGCGAGGCTGTGTCCCCGCCGGGTGAATCTCAGG
TGTCCCTCCCAGCCAGAGGCCTGGAACCCTAGCGCTACCTCCCAGTGGCCCATGCACTCGCCAGGGGCTG
CAGACAGCCTGGCTGCTGAGTGGGCCCAGTAGGAGACTCGGGGGCAGCTGGATGGGGGCTTTGAGGGCCA
GTACTTGGGGTGTGGCCACCAAGGCCCTGGCTCAGTCCGACGGGGGCGGGGCTCACGTGCCTTTGCCCTT
GTGCTACGCAGCCCCGTGTGTGGAAGCCGCCTTGCGCCAATGGGCACCGACCCGTGGGCTGAGGGAAACA
CTGCGCCCCGTGGCCTCCTCACCACCCACAGCTCCCAGCTGCACGCTGCCGCCCAGGGCTCCAGGAGACG
GTACTGTGGGACGCATCTGTCTTTGTTGCAGAGCCTCTGCCTGTCTTTGGGGGTTCCTGGATGGAGGAGG
CCCACCACCCTGTACCCCTGGCGCCGGGCGCCTGCCCTGAGCCGCGTGCGGAGGGCCTGGGTCATCCCCC
CGATCAGCGTATCCGAGAACCACAAGCGTCTCCCCTACCCCCTGGTTCAGGTGAGCAGGTGGAGGGGGCA
GGAGGGGAGAAAGGGGTAGGCTGGTCCCCAGTGGGCCTCCCTCATTCTCTAAAGGTCTCCTGGGAGCCAG
CGGGGCCCCATTTCAGGACAGAGCTGAGGCAGGACTCGCCCACCTTGCCAGGGCTCTGGGCTTCCAACCT
GGGTCTAGAGCAGGGCTCAAACCCTCCCCATTGCCCCAGGCCACAGGGGAGTCCAGAGCTGCTCCCACCC
CACATGCGCCTCGGGTGGACATACTCCACGTTAGGCTGCACGCTCGGGCGTGATTTTTGTGTGCGTGTTC
GCCGACGCTGAGTAGATGGAGGTGAAGGTGAACTGCCTTGCGTGGGCCTCAGCTTCACATACCCAAAATC
GCCCCAGAGACCAGGGCCCTGAGGACACCAGGAGCTGAGCCTGAAATGAGGCTGCAAGACGGGGCACCCT
TGGGGCCACAGGCTGAGCTGTCCCCAGCCCAGCACAGCTCCTCATTGGGTACCAGGATCCGTCCTCCAGT
CCTAGGAGACTTAGACCTGCCCTGCTGTCAGCTGGGGAGGGGCCTGAGGGGCTGCAGAGAGGGCAGAGGC
CCCCGCCCGGAGCAGCTCTCCCCAAACCCATTTCCTGCCCCACAGATCAAGTCGGACAAGCAGCAGCTGG
GCAGCGTCATCTACAGCATCCAGGGACCCGGCGTGGATGAGGAGCCCCGGGGCGTCTTCTCTATCGACAA
GTTCACAGGGAAGGTCTTCCTCAATGCCATGCTGGACCGCGAGAAGACTGATCGCTTCAGGGTGCGGAGC
TGCGTGGTCGGACCTGTGCCCCTCAAGCAGGCCTGGTGGAAAGCCAGTGCCCCTCCTCCCCACCAGGCTT
CTCCTCCCCGCTCGGTGGGCTTCAGGCCAGACTGCAAGATCCAGGCACCCTTAAGTGAGGGGGCAGGTAC
AGGGGTTTGGGACCGAGGCCCTGCAGCCGGAGGAGGCAACTGTTGAGGGTTATGTGCCCATTTAACTGGA
GGGCAGACAGAGGTCCCGTGGAGGAGCGGCTTGGTTCTGCCAGGGAGGAGCATGCTGCAGCCGCTGTAGC
TTCTGTGGCTCAACCACAGCCAGCAACCCACTGGTCCTGGGAGCTCAGGAGTGTCGTGAAGAATCTCTAA
ATAGTGTTGTTTTTGTTTTTTGTTTTTGAGATGGAGTCTCGCTCTGTCGCCCAGGCTGGAGTGCAGTGGC
GGGATCTCGGCTCACTGCAAGCTCCGCCTCCCGGGTTCACGCCATTCTCCTGCCTCAGCCTCCCGAGTAG
CTGGGACTACAGGCCCCTGCCACCATGCCCGGCTAATTGTTTGTATTTTTAGTAGAGACGGGGTTTCACT
GTGTTAGCCAGGATGGTCTCGATCTCCTGAGTGAGCCACCGCGCCCGACCTTTTTTTTTTTTTGAGATGG
AGCTTCACTCTACCGCCCAGGCTGGAGTGCAGTGGCACAATCTCAGCTGACTGCAATCTCTACCTCCCCA
GTTCAAGCAATTCTCCTGCTTCAGCCTCCTGAGTATCTGGGATTACAGGCTCCACCACCGTGCCCAGTTA
ATTTTTGTATTTTTAGTAGAGATGGGGTTTCACCATGTTGGCCTCCCAAAGTGCTAGGATTACAGGCGTG
AGCCACCACACCTGGCCCCAAAATAATGTTGATCCCAGTGGCCGCCACTGTTCTGGGCAGTGGGGTTAGT
CAGGGAAGAAACCACTGATGCCCCTTGGGGGCTGGGATTCTCAGAGAAGGGGCAGTGAGCGCCGGGCGCG
GTGGCTCACACAATCCCAGCACTTCGGGAGGCTGAGGCAGGAGGATCGCTTGAAGCCAATAGTTCGAGAC
CAGGCTGGGCATCAAAGTGAGGCCTTGTCTCAACAAAAGATAAAATGGTAAAACAAAAAAGATAAAATGG
TAAAATAAATTAGCCAGCGGTGTTGCTGCGTGCCTGTGGTCCCAGCTGCTTGGAAGGCTGAGGTGGGAGG
ATCCCCCGAGAGGTCGACACTGCCGTGCGCCGTGATTGCGCCGCTGCGCTCCAGCCTAGGTGACAGAGGA
AGACCCTCTCTCAAAAAAAAAAGAAGAAAAGAGGACCGGGTGCGGTGGCTCACGCCTGTAATCCCAGCAG
TTTGGGAGGCCAAGGCAGGCAGATCATGAGGTCAAGAGATCGAGACCATCCTGGCTAACATGGTGAAACC
CCGTCTCTACTAAAAAATACAAAAGTTAGCTGGGCATTCTAGCGTGCGCCTGTAGTCCCAGCTACTCAGG
AGGCTGAGGCAGGAGAATCGCTGGAACCCGGGAGGCGAAGGTTGAAGTGAGCTGAGTCCTCGCCACTGCA
CTCCAGCCTGGTGACAGAGTGAGACTCTGTCTCAAAAAAAAAAACAGAAGAAGAAAGAAGAAGAGGAGGA
GGAAGAGGAGGAGAAGAAGAAAGAAGAAGAAGAAAAAAAAGGGCAGTGGGAGCAAGCCCTAGAGCCCAGA
AAACCAGAACCCCGTCTGCACTCCCTTCAGGCGCCTGCCCTCCCCCAGCCTCCCCTCCCCCAGCCTGCCC
TCCCCCAGCCTGCCCTCCCCCAGCCTCCCCTCCCCCAGCCTGCCCTCCCCCAGCCTCCCCTCCCCCAGCC
TGCCCTCCCCCAGCCTGCCCTCCCCCAGCCTCCCCTCCCCCAGCCTGCCCTCCCCCAGCCTGCCCTCCCC
CAGCCTGCCCTCCCCAAGCCTCCCCTCCCCCAGCCTGCCCTCTCCCAGCCTGCCCTCTCCCAGCCTGTCC
TCCCCCAGCCTGCCCTCTCCAAAATGTCATTTTGGGCTGGGCACAACGGCTCATGCTTGTAATCCGAGCA
CTTTGGGAGGCCGAGGCGAGTGGATCACCTGAGGTCAGGAGTTTGAGAACAGTCTGGCCAACATGGCAAA
ACCCCATCTCTACTAAAAATACAAAAAATTAGCCGGGTGTGGTGGCACGCGCCTGTAATCCCAGCTAGTT
GGGAGGCTGAGGCAGGAGAATCACTTGAACCCAGGAAGTGGAGGTTGCAGTGAGCCAAGATCACACCACT
GCACTCCAGCCTGGGCAACAGAGCGAGATGCCATCTCAAAAGGTAAAAAAAAAGAAAAGTAACTTTGTAC
TTGATGTGGGGAAAGGACGCTTCCTGCCTTTGCCTCTGCAGGAAGCAAAATTTCCTTTTATTTGCCCCAA
ATCCTCCTTTAGACTTTGAGAACCAGTCCTTGTATCCCTTACATCAAAAGTTATTAGAGAGTATTTCTCA
CTTGGAAACTGTAATTGTGCCCATTCTCCGAAAGGATGGGAGGCAGTTTAAAATGTCATGTGCACAGGGG
AATTAAACTTCAGGAGGAGGCCAGGCGCAGTGGCTCACCCCTGTAATCCCAGCACTTTGGGAGGCCGAGG
CGTATCACCTGAGGTCAGGAGTTTGAGACCAGCCTGGCTAACATGATGAAACCTCATTTCTACTAAAAAT
ACAAAAAATTCGCTGGGCGTGGTGACGTGCACCTGTAATCCCAGCTACTTGGGAGGCTGAGGCAGGAGAA
TCGCTTGAACCCAGGAGGCAGAGGTTGCAGTGAGCTGAGATGTCGCCATTGGACTCCAGCCTGGGCAACA
AGAGTGAAACTCTATCTCAAAAAAAAAAACAAAAAACAAAAACATAACTTCAGGAGGAACAAAGACCACA
GAGAGACAGGGAGAGAGATTAGGAACCCTCTGAGAGCCTTACTTTAGTCACACGTGAAGCTGTGAGCTTC
CTGGCTGCCAAGGCAAGCAGGGAAAGTGGCACCTGCCCTGTCTGCTAAGGGAAGGTGGCTCCCGTTCAGG
GACGTCCTCAGTCACTGTGACAGATGCCCCACCCTGTGCTGTTTCTCGCCTGTTTAAGCTGGTGTTTCCA
GCTGGAGACAAAGTCTGAACGTGCTGTCTCTTTCGCATGGCCCTAACGGGAGCCACAGAAATTTGGGGGC
CACAGAGCCAGCCCTTGCTCTATGTTTGAACAGCTAAGAGCGTTTGCCCTGGACCTGGGAGGATCCACCC
TGGAGGACCCCACGGACCTGGAGATTGTAGTTGTGGATCAGAATGACAACCGGCCAGCCTTCCTGCAGGA
GGCGTTCACTGGCCGCGTGCTGGAGGGTGCAGTCCCAGGTGAGACAGGACCACAGCCCCGGGCCGGGAGG
GGCTGCAAGGAAGGGCTCTGTGAAGTCCAGGACTTCCCTTAAGCAAGAATTCCAGAGGCCCCTCAGAGTC
TAAAAATAAGTAAACAAGTCTCCGAGGCAGGTCCGTTTCCACAGGTCAACTCCTGCCACTTCCCATTCCA
ACATAAATTTCAAACCCAAAAATCTGAGCCTGGGAGTGGAGGTTTTTCAGGGGTCTTGTTCTCCCCCAAA
AGCAAATGACTTCTCCCTCCTGCTGAGGGGGCCATCTGGAGCCAGGCACACTCCATCCTCACCCTGTACT
CACATGGGGTCCAGAGCACCCACAGGCATCTCTAGGTTGTGATTCACCCTAAACCCAGGCTGACCTGTAA
GCTCCAGCCACGGGGACTCGCTCCCACCTCCCTGGGGGCCGGTGGTAAAGACCTGGTGGAAAGGCACAGG
CCCCCTCGTGTCTGAGGCTACCAAGGACTGTGGCCAGGCCTGGCCCAGGCTCAGGCTTGGAGGCTCATAT
CCCGCCTCAGTTTCCTTCCCGTGTCTGTGGCAGTGGCCTCCGCGCCTCGTTCTGCCCCAGGTGACAGACA
GCACGTCCTAGCTCCAGCTGTCCCGGGGGGAAGTCACAGCCAGCCAGGAGCTGCTCCCCAGGGTGGCCCA
AAGCCCGTCCTTCCCACCCCACCTCGGTGGCTAAGTCCACCTGAGCTGCCAGAGGGGTCAGGCCTAAATC
TCAGGAGAGGGAAATGGGGGGAGGTGAAACCTGGTCCTGCCATATGTGGTCCCTCTAGCGGCACCTTCGT
GAGTACCGCTTCCCACAGCAGGTGGGGCGGGGGCTCTGGCCTACACAGTCACAGTGCGCGTCCTCTGCTC
GGCTGTCGTGTGTCCTGCACCCAGCCTGCCCGCGCATCCTCTGTTTTATCCTGCCCTTGCATCCTCTGTC
TTATCCTGTCCGTGCGTCCTCTGTTTTATCCTTCCCCTGCGTCCTCTGCCCAGCCTGCCCGTGCGTCCTC
TGTCTTATCCTGCCCGTGTGTCCTCTGTTCAGCCTGCCCATGCATCCTCTGTCACCCTGGATGCCAGCAG
CTCCTGGAGGCCCCCGCTAGCTAGCCCAATCTGACCCAGCGCCCTTAGCCCCTGCACTGGGCAGGCCCAG
GAGCCAGCACCTGACCCAGGTCGCATCCAATGCTCCTGTGCTCCGACCCCATAAACAGCACAAGCCGGGC
AAAAGGCTTCGACCACATAGGTGGCCTGGACACAGCACGGTGGGCTTCAGGGGCAGCGGGAAGCATCTCG
ACCTGGGGGTTGCCGCATGCTGGCCGCCTCGGTGACGCAAACAGCAGCATGGACTGACTGCCCCATCTGG
GGTCAGCCGTGCTGGAACCGGGGAGCCTGTGCACACGAGGTGCCCCCACGCCCCTCACAATGCCCCCAGC
CCATCGGCCCTGTGGACGTTGGCCCTCACGCCTCCCTGTGCTTCCCAGGCACCTATGTGACCAGGGCAGA
GGCCACAGATGCCGACGACCCCGAGACGGACAACGCAGCGCTGCGGTTCTCCATCCTGCAGCAGGGCAGC
CCCGAGCTCTTCAGCATCGACGAGCTCACAGGAGAGATCCGCACAGTGCAAGTGGGGCTGGACCGCGAGG
TGAGGTGGCGCCCCGGCAGCTCCACACCCGCACGGCCAGGGCAGCCCATCTCCTGCGGGTCCCTCTGCCC
CCAGCCTGCCCACCCCAGACGCTCCTGTCCCTGCCGTCACTGCAGAGCTTGCAGTGGCCCTGGCTCCTGA
GGAGATGGCATGGGGTGAACCCACTGATGCGCAGACAGGAGCCGCGCTGGCCCGGGTGGGAGGGGTCTGG
TGCAAGTAGGGCGTGAGGGGCCCGGCACAGGGCAGGGCTCCCCAAGCAGCCCCGCCCCTCTCAGCCTCAT
GGCAACGGCTCCCCGCTGGTAAGTAACTAACTCCTTCTGTCAGGGCCCCTGTTGCCCCTTGCTGTCGGAC
TTCGGTGCTCCCGGAAGACCCCCCTTGAGTCAGCTCCTCTAGGAAGCCCTCCTGGTTACACTGTTACGTG
GTCTTTGTCCATTTCTGTTCCTGCCACTAACCACGGTTCCCCAGGGCAGGGACCCAAGCTCACTGGTGTG
CTAGGTGCCCAGCACAGAGTAGGTGCTCTGTAAATGCTTACCCAGCGCACAGTAGGTGCTCTGTAAACAC
CCAGCGCACAGTGGTGCTCTGTAAACGCTTATCCAGCACACAGTAGGTGCTCTGTAAACGCTTACCCAGC
GCAGAGTAGGTGCTCTGTAAAGGCTTACCCAGCGCAGAGTAGGTGCTCTGTAAATGCTTACCCAGCGCAC
AGTAGGTGCTCTGTAAACACCCAGCGCACAGTGGTGCTCTGTAAACGCTTACCCAGCGCACAGTAGGTGC
TCTGTAAAGGCTTACCCAGCGCACAGTAGGTGCTCTGTAAACGCTTACCCAGCACACAGTAGGTGCTCTG
TAAACGCTTACCCAGCGCACAGTAGGTGCTCTGTGAACACCCAGCGCACAGTAGGTGCTCTGTAAACGCT
CACCCAGTGCACAGTAGGTGCTCTGTAAACGCTCACCCAGCGCACAGTAGGTGCTCTGTAAACGCTCACC
CAGCGCACAGTAGGTGCTCTGTAAAGGCTCACCCAGCGCACAGAAGGTGCTCTGTAAAGGCTCACCCAGC
GCACAGTAGGTGCTCTGTAAACGCTCACCCAGCGCACAGTAGGTGCTCTGTAAAGGCTCACCCAGCGCAC
AGAAGGTGCTCTGTAAAGGCCCACCCAGCGCACAGTAGGTGCTCTGTAAACGCTTACCCAGCGCACAGTA
GGTGCTCTGTAAACGCTTACCCAGCGCACAGTAGGTGCTCTGTGAACACCCAGCGCACAGTAGGTGCTCT
GTAAACGCTCACCCAGCGCACAGTAGGTGCTCTGTAAACGCTCACCCAGCGCACAGTAGGTGCTCTGTAA
ACGCTCACCCAGCGCACAGTAGGTGCTCTGTGAACACCCAGCGCACAGTAGGTGCTCTGTAAAAGCTCAC
CCAGCGCACAGAAGGTGCTCTGTAAACGCTTACCCAGAGCACAGTAGGTGCTCTGTAAACGCTTACCCAG
CGCACAGAAGGTGCTCTGTAAACGCTTACCCAGCGCACAGAAGGTGCTCTGTAAACGCTTACCCAGCGCA
CAGTAGGTGCTCTGCAAACACCCAGCACACAGTAGGTGCTCTGTAAACGCTTACCCAGCACACAGTAGGT
GCTCTGTAAACGCTTACCCAGCACACAGTAGGTGCTCTGTAAACGCTTACCCAGCGCACAGTAGGTGCTC
TGTAAACGCTTACCCAGTGCACAGTAGGTGCTCTGTAAACGCTTACCCCGGGCACACAAGGTGCTCTGTA
AACACTTACCCAGCACAGAATAGGTGCTCTGCAAACGCTTACCCAGTGCAGAGTAGGTGCTCTGTAAACG
CTTACCCAGCGCACAGTAGGTGCTCTGTAAACGCTTACCCAGTGCACAGTAGGTGTTCTGTAAATGCTTA
TCCAGTGCCATAGGCTAGGGCAGCATCACAGCCTCATGCACTTAGGATCAGGGCAGGATTCTCAGGGCCA
CTTGGGGTCTTCAACACCCACTGGGTGCTCCCGTAGGAACTGAGCTGGCCAGGTGGCCTGGCTCCCACCC
CTGACCAGTCCCCATGTGCCCCACCTGGGCCCTCATCTTCTGACCCTGTGCCCCACATCCCCAGGTGGTC
GCGGTGTACAATCTGACCCTGCAGGTGGCGGACATGTCTGGAGACGGCCTCACAGCCACTGCCTCAGCCA
TCATCACCCTTGATGACATCAATGACAATGCCCCCGAGTTCACCAGGGATGAGGTGCTGCTGCTGTCCCT
CCCTCGAAAGTAGCCCCTGCTTAGAGCTGCCTTCCCTTCTTGGGCTCCTGCAGAAGGCAGCGGGCTTCAT
GATGGGGCAGGAGGATGGTGTGCTTTGGAGAAGGAACTCCGCGTGGGCGGGTGGAAGCCCAAGCTGGAGG
GGCCCCGGGGTGCTGCGTGGCAGTGTGTGAATGGAGTCAGAGTTGGGAGAGAGGCCCTTGGGGAGAAGCA
GCCCATGGGACCCTCTGAGGCCCTCTCGCATTGCAGAGCAAGTCTCCCTCCGGCCTTTGCTGTATGGGAA
GGGAGCACGAAGCTGAGCCCACAGAGGAGGACCATGGGGGCACGTGGGGGACAGTGACTCACAAAACAGA
CAAACCTGAGGACACCCAGTGGGTGGGGAGCAGCTTCGACAGGAGCAAGGGAGGGTGTTCCTGCTGGGAG
GCAGGAGGGAGGGTGGGAGGGGAGGGTGGGCTGAGGGCCCTGAGGGCCCCACCCATGCTGTCCCCCCAGC
CCTGCTGGTAACTGGGGCTGGGATCCCCCACCCAGTTCTTCATGGAGGCCATAGAGGCCGTCAGCGGAGT
GGATGTGGGACGCCTGGAAGTGGAGGACAGGGACCTGCCAGGCTCCCCAAACTGGGTGGCCAGGTTCACC
ATCCTGGAAGGCGACCCCGATGGGCAGTTCACCATCCGCACGGACCCCAAGACCAACGAGGGTGTTCTGT
CCATTGTGAAGGTGAGCGGCCCCCGGCTGGCACACAGATGCCGGCAGACGCAGATGCCGACACACACAGA
TGCCCACACACAGATGCCGGCACACACAGATGCCCACACACAGATGCCGGCACACACACATGCCGGCACA
CACAGATGCCCACACACAGATGCCGGCACACACACATGCCGGCACACACAGATGCCCACACACAGATGCC
GGCACACACAGATGCCGGCACACACAGATGCCCACACACAGATGCCGGCACACACAGATGCCCACACACA
GATGCCGGCACACACACATGCCGGCACACACAGATGCCCACACACAGATGCCCACGCACAGATGCCGGCA
CACACAGATGCCCATGCACAGATGCCGGCACACACAGATGCCCACACACAGATGCCGGCACACACAGATG
CCCACGCACAGATGGCGGCACACACAGATGCCGGCACACACAGATGCCGGCACACACAGATGCCCACACA
CAGATGCCCACACACAGATGCCCACGCACAGGTGCCCACACACATGCTGGCACACAGATGCCCACGCACA
GGTGCCCACACACAGATGCCCACGCACAGGTGCCCACACACAGATGCCAGCACACACAGATGCCCACACA
CACATGCCCACACACAGGTGCCCACACACAGATGCCCACACTGTCCACACACCGATGCTGGCACACATAG
ATGCCCACACACAGACACCCACACACAGATGCCCACACACAGGTGCCCACACACAGATGTTGGCACACAC
AGGTGCCCACACACAGATGCCGGCACACACAGATGCCCACACACAGATGCCCACACACAGATGCCCACGC
ACAGGTGCCCACACAAAGATGCCGGCACACACAGATGCCCACACACAGATGCCCACACACAGATGCCGGC
ACACACAGATGCCCATACACAGATGCCCACGCACAGGTGCCCACACACAGATGCTGGCACACACAGATGC
CCACACACATGCCCACACACAGATGCCGGCACACACAGATGCCCACACACAGATGCCGGCACACACACAT
GCCGGCACACACAGATGCCCACACACAGATGCCGGCACACACAGATGCTGGCGCACACAGATGCCCACAC
ACAGATGCCCACACACAGATGCCCATGCTGGGGTTTGAGCCCCTTCCTGGCAGGTTCACATCCTCTGACC
AGCTGGGACCTTGTGTTCGTCCTTGGGTGAGAGAGAGTGGGGGAGGGCATGGAGCCCCCCAGCCTGAGGA
CTGGGTCCCAGGCCACGCTACCCATGGGGCTTGAGGGCCAGAGGCAGGGGACACCCTCCACCACTTTGCT
CTGTCCAGCCTTCCTCCTTTCATATGGAATCCATGTACAAAATCACAGCTGCACACCTGTGTCAGTCCTG
GTTCTACCAGAGAAAGAGGCCATAGGAGACACATGCACAGATATGTCCACACGTGTAGATGTGGGTGTAT
TTAGATACTGACACAGTAAGAGGTTTACTGCAAGAAATTGGCTCATGCGGTGCTGGGGTCTGGCTGGGCA
GGTCTAACGTGCAGACCACAGGCTGGAGCCGACGCAGCAGTTTAGAGGCAGAGAAAATGCAGTTTTGCTC
TCAAAGCCTTCACCTGAGTGGATAAAGCCAACCCACATCACCCAGGACGATCTCCTTTATTTAAAATCAA
GTGACTGCAGGGGCTGGCGCCATCCCCCAACGGCCCTCGCTGAAATACCCGCGGAGTATGTGCTTGAACC
CTGGGCTGCTCCCCAACCACCACCACACACAACCAGCATTTTAATAATATTCTTTGTGCCTTCTTGTCCT
GCATAATTTGTTTTTTCAAGGTTAAAAAGGGGCAGAAGGAAAGTACAGGTGCTCTGGACAGACCCAAGGA
GTGGCTCCCCCATGGCACCTGCCCTCGGGTGCCCACACTTGCGTTGGGCGGATGACGGGCTGTGCTTCCT
TCCCTCAGGCCCTGGACTATGAGAGCTGTGAACACTACGAACTCAAAGTGTCGGTGCAGAATGAGGCCCC
GCTGCAGGCGGCTGCCCTTAGGGCTGAGCGGGGCCAGGCCAAGGTCCGCGTGCATGTGCAGGACACCAAC
GAGCCCCCCGTGTTCCAGGAGAACCCACTTCGGACCAGCCTAGCAGAGGGGGCACCCCCAGGCACTCTGG
TGGCCACCTTCTCTGCCCGGGACCCTGACACAGAGCAGCTGCAGAGGCTCAGGTGGGGCTCCTGAGGCCC
TGGGAGAGGTAGAGGAGGCTAACGGCCCCATTCCTGCTTCGGGTGCCCCTGATCCCTGGGCTCTGAGGGT
CAGAGGGTGTAGGGGCCATTTGCTGTGTGGGTTCCTGAAGGTCTGGAGGGTCTCGAGTCCCGAGCATGCC
CGTGTGTGCATGTTGGGGTGACCTCACCCCCTAGGGCAGGCCCAACTCCAGCCTGTGCACGCCAGTCTGT
CCCTGCCAGCCGTGTCCATCCCAGGAGGCCCTTGCTCCCAAGGGACTGGCCGTTCGCATCTCATTTCACT
CCAAGCCTGGCCCTGTGTATCCCTGTCTGTGTCCACGGGGCCCGGGAAGGGCCAGCACCAACTGAGGCTT
AACTAGGTTGCCACACAATTGTCTCAGCGCCCTGGACAGCTCGCCTGCCCTCCCTGAGCCTCAGTTTCCT
CACCTCACAGAAGGTGCTGGTGACTGCAGCTCCCATCATATTCACATGGGTTGTGTGTACATGTGTATGT
GCATATGTGTGTGCACATGTGTGGTACATGCGTGTGGTGTATAGGTGTGTGGGGGGTGGTATGTGCAAGG
TGGGGGGGAGTGTCTATGTGTATATATGTGCATGTGTGTGTGCTGTGCATGCCCATGTATGTGTATGTGG
TGTGTGCATGCATGTGGCATGTAAGGCTGTGTGTGCGTGTGTATATGTTGTGTGCATGTGTGCATGCATG
TGGTATGTATGTGTGTGCCTGTGTGTGTGCAGTGCATGTCACGCACCCATACCTGACCACACCTGTGGGG
CCCTGGGGTAAACTCAGATCCCACTCTTCCCCTCCCCTGCATCAGCTACTCCAAGGACTACGACCCGGAA
GACTGGCTGCAAGTGGACGCAGCCACTGGCCGGATCCAGACCCAGCACGTGCTCAGCCCGGCGTCCCCCT
TCCTCAAGGGCGGCTGGTACAGAGCCATCGTCCTGGCCCAGGATGACGGTGAGCGGCGCCGCCGGCTTGG
GGCTCCCTGACCTGGCCTTGTCCCGGCTGAGCACCCCTGCCAGTGTCGGAGGGCTCTGCCCATGTCGCCC
GGGGGCTCAGAGCTGCGCACCCGCTCTGAGCCGACTGGTGGGGCAGGCTGGGGTGTTGGGGTCACTAAGC
CGCGGCCTCCTCGCCTGCAGCCTCCCAGCCCCGCACCGCCACCGGCACCCTGTCCATCGAGATCCTGGAG
GTGAACGACCATGCACCTGTGCTGGCCCCGCCGCCGCCGGGCAGCCTGTGCAGCGAGCCACACCAAGGCC
CAGGCCTCCTCCTGGGCGCCACGGATGAGGACCTGCCCCCCCACGGGGCCCCCTTCCACTTCCAGCTGAG
CCCCAGGCTCCCAGAGCTCGGCCGGAACTGGAGCCTCAGCCAGGTCAACGGTGCGCTCCCCTCACCGCCG
CGCTCCCCCCATCCCCACGCTCCCCCCACCCCCACATTCCGGCCTCGGACGGGGGCAGGAGGGTGAGGGG
CATGCAAACCCGTGGTCCTGCAACAGGTCCCCTCCCGCCACCCCCCCCACCACTGCATCCTCCCGTGGGG
CAGGGTTACTCATTGTGCCCAGAGGACGGTGGGGGTGGGGGGGACCCAGGCCCAGGATCTCGGGATCCCC
ACCCTGTCTCGGCGCGAGGAGGGCAGGCGAAGTGGGGGCGGCCTCGGGAGGCCCTCGCTCACCACAGGCG
CCCTCCGCAGTGAGCCACGCGCGCCTGCGGCCGCGACACCAGGTCCCCGAAGGCCTGCACCGCCTCAGCC
TGCTGCTCCGGGACTCGGGGCAGCCGCCCCAGCAGCGCGAGCAGCCTCTGAACGTGACCGTGTGCCGCTG
CGGCAAGGACGGCGTCTGCCTGCCGGGGGCCGCAGCGCTGCTGGCGGGGGGCACAGGCCTCAGCCTGGGC
GCACTGGTCATCGTGCTGGCCAGCGCCCTCCTGCTGCTGGGTGAGTGAGCGCCCCGCCTCCACCTGGACC
CTCGGACCCTCGGACCCTCCTCCCCAGGCCGTCCCCTGCTAACCAGCCACGCCGCTTCCTCCCCAGCTCC
GCCTCCTCCCTAACCCCGCCCCCTCATTACCAGCCACGCCGCTTCCTCCCCAGCTCCGCCTCCTCCCTAA
CCCCGCCCCGTCATTACCAGCCACGCCGCTTCCTCCCCAGCTCCGCCTCCTCCCTAACCCCGCCCCCTCA
TTACCAGCCACGCCGCTTCCTCCCCAGCTCCGCCTCCTCCCTAACCCCGCCCCCTCATTACCAGCCTCGC
CGCTTCCTCCCCAGCTCCGCCTCCTCCCTAACCCCGCCCCCTCATTACCAGCCTCGCCGCTTCCTCCCCA
GCTCCTCCTCCTCCCTAACCCCGCCCCTCAGTACCAGCCACGCCGCTTCCTCCCCAGCTCCTCCTCCTCC
GTAACCCCGCCCCCTCAGTACCAGCCACGCCCCTTCCTCCCCAGTTCCACCTCCTCCGCAACACCACCCA
CTCATTACCAGCCACGCCGCTTCCACCCAACCCGCCCCCTGCTCGCCAACCCCGCCCCCTCATTACTAGC
CACGCCCCTTCCTCCCCAGCTCCGCCTCCTCCCTAACCCCGCCCCCTCATTACCAGCCACGCCCCTTCTC
CACTGCCGCCTCCTCCCCAACACCACCCACTCATTACCAGCCACGCCGCTTCCACCCAACCCGCCCCCTG
CTCGCAAACCCCGCCCCCTCGTTGCCAGCCACGCCTCTTCCCCAAGCCGGCCTCTCCTTACCAGCCAAGC
TCCCTCCTCCACAACCCGGCCCCCTCCTCCCTAACCTCGCGGCTTCCTCCCCAACCCCGGCTCCTCCATG
CCAGACACGCCCTTTCCCCAACTGCCGCCCCCTCAACCCCACCCCTGCTTACCAGCCTTGCCCCGCCCCG
CCCCCTCCTCCCACCTCCTTCGCAGCCCGGCCCCCTGAAGTCGCGCCCTGTGCCTGGCCCCAGCCTGCGT
CCCCTCATTCCCCAGTGCTGGTCCTGCTCGTGGCACTCCGGGCGCGGTTCTGGAAGCAGTCTCGGGGCAA
GGGGCTGCTGCACGGCCCCCAGGACGACCTTCGAGACAATGTCCTCAACTACGATGAGCAAGGAGGCGGG
GAGGAGGACCAGGTGAGGGGGCAGGTGTGGGTGGGGAGGGGTCCCCAAGGAACCCAGGTCGCGGGCCTTC
TTACAACAAGCTGGCCAGGAGCCTGTACCTGAGACCTCCACCAGGGCCACCCGAGGGATGCCTGGCTCTG
TTCCACCTCCTCGCCCACAGGACGCCTACGACATCAGCCAGCTGCGTCACCCGACAGCGCTGAGCCTGCC
TCTGGGACCGCCGCCACTTCGCAGAGATGCCCCGCAGGGCCGCCTGCACCCCCAGCCACCCCGAGTGCTG
CCCACCAGCCCCCTGGACATCGCCGACTTCATCAATGATGTAGGTGCTCCTGGGGACACCCCAGTACACA
CAGGCACGCACAGGTGCACACACACATGCACATGTACACACCTGCACATGCATGCAAGAACCGGCGCCTG
CATGCACTTATGGGCCGTCCCAGAGCACCGCAGAGGAAGATGGTGTGCGGGCGGGAGTGTGGAAGCCCCG
CTGCCACCGTCCAAACTGGGCCCTCAGCCTCCACCCGCGACGCGGGTCTTTGCACAGATGGAGTGGCGAT
GGCCACAGACCCATCCACTGCCCCGTGTCCACCATGGAGGGGGTGGGGGAGCCCCGGGAGCATCCCCTGG
GCTTCGGTGGCTGTCAGCGGAATCAGGGCCTCCATAGAAGACCCTCTGGACACAGCGTTAATGATTCCCT
GCCATCCACAGGATGAATGTGCACACGGGGAATCTGGGGTGTGGGGCGCACAGGGAACCCTCCTCTGTGC
CTGTCTGCACCTGTAGCACCTGGCTCAGAGAAAGCCCCCAGCCAAGGTGTACACCCCCGGTTAGGAGCGT
GTGTCCCCACGAGCTGAGAGGACAGACATGCAGACAGAGCCTCCGAAAGGCAGACGGGCCGTTCTGCGGA
TGGGGGTGTCCAGGAGGCTCGGGTTCTGCCTTGAGTGGACAAGGGCCACGGGGAGGAAGAGACCAGGGTG
AGGGGGAGCAACCCCTTCCCACCTTGCTGGGGCAGGGCAGAGCCCCGGGGGACGGGCTGGCCGCTGGCCT
AATACTGAGGAAGGGGGTAGTCCGTGGCACCCTCTTCACAACCCTGCTGCTGTGCCCTCAGGACGCTTTG
CCTCCCCTCAGACCTCGCCCCCGGACGTGGGCAGCTTCCCCTTCCTGAGCCCGTGCCTTGAGCTGACTTT
GCCCTGGGCTGTGGCCACGGCGGTGGGGCACCCGCTGGCCCCTCCATGTGTCTTGAGCTCTCCGGGCCTC
TTTATAGGGCTTGGAGGCTGCAGATAGTGACCCCAGTGTGCCGCCTTACGACACAGCCCTCATCTATGAC
TACGAGGGTGACGGCTCGGTGGCGGGGACGCTGAGCTCCATCCTGTCCAGCCAGGGCGATGAGGACCAGG
ACTACGACTACCTCAGAGACTGGGGGCCCCGCTTCGCCCGGCTGGCAGACATGTATGGGCACCCGTGCGG
GTTGGAGTACGGGGCCAGATGGGACCACCAGGCCAGGGAGGGTCTTTCTCCTGGGGCACTGCTACCCAGA
CACAGAGGCCGGACAGCCTGACCCTGGGGCGCAACTGGACATGCCACTCCCCGGCCTCGTGGCAGTGATG
GCCCCTGCAGAGGCAGCCTGAGGTCACCGGGCCCGACCCCCCTGGGCCTGGGGCAGCCTCCTTCCTGTAG
GCGAGGGCCCAAGTCTGGGGGCAGAACCTGAGTGTGGATGGGGCGGCCAGGAAGAGGCCCCTTCCTGCCG
GGGTGGGAAGAGTTTCTCTCCATCGGCCCCATGCGGGTCACCTCCCTAGTCCCACCTTTGCCTCCTACCA
GTGAACCTCATCTTTGTATGAAAGACAGCAACCTCCTGGGTAAATCTGAATGAAAAACGTGCTAGTCTCT
TTCATGCA

1. A method of treatment and/or prophylaxis of a condition associated with T-cell mediated chronic inflammatory disease, including T-cell auto-reactivity, T Cell mediated chronic inflammatory disease and autoimmune disease, the method comprising administering an agonist of CDH15 to an individual in need thereof, wherein the agonist is not PEPITEM or an analogue thereof.

2. The method according to claim 1, wherein the condition is selected from the group consisting of: diabetes mellitus (type I); juvenile onset diabetes; multiple sclerosis; rheumatoid arthritis. Chrohn's disease; artherosclerosis; psoriasis; inflammatory and fibrotic liver disease(s) including steatohepatitis and cirrhosis; Sjogrens Syndrome; Ischaemia reperfusion injury; transplant rejection and uveitis.

3. The method according to claim 1, wherein the condition is selected from the group consisting of nephropathy; diabetic kidney disease; peripheral neuropathy; diabetic retinopathy; and cardio-cerebral disease.

4. The method according to claim 1, wherein the agonist of CDH15 is an antibody specific for CDH15.

5. The method according to claim 1, wherein the agonist of CDH15 is an antibody specific for a protein encoded by the CDH15 gene.

6. The method according to claim 5, wherein the protein encoded by the CDH15 gene is a spliced gene.

7. The method according to claim 4, wherein the antibody binds to the whole or part of an amino acid sequence corresponding to SEQ ID No. 2, or a homologue thereof.

8. The method according to claim 4, wherein the antibody is one selected from the group consisting of: Rabbit anti-human CDH15 polyclonal antibody IgG-ab75626 (Abcam); Rabbit anti-human CDH15 polyclonal antibody IgG clone H-71-sc-10734 (Santa Cruz); Rabbit anti-human CDH15 polyclonal antibody-SAB4500040 (Sigma-Aldrich); and Sheep anti-human CDH15 polyclonal antibody-AF4096 (R&D systems).

9. The method according to claim 4, wherein the antibody is a monoclonal antibody.

10. (canceled)

11. (canceled)

12. The method according to claim 5, wherein the antibody is the one selected from the group consisting of Rabbit anti-human CDH15 polyclonal antibody IgG-ab75626 (Abcam); Rabbit anti-human CDH15 polyclonal antibody IgG clone H-71-sc-10734 (Santa Cruz); Rabbit anti-human CDH15 polyclonal antibody-SAB4500040 (Sigma-Aldrich); and Sheep anti-human CDH15 polyclonal antibody-AF4096 (R&D systems).

13. The method according to claim 5, wherein the antibody is a monoclonal antibody.
